(19) United States
(12) Patent Application Publication
(10) Pub. No.: US 2009/0175798 A1
(43) Pub. Date: Jul. 9, 2009

(54) Mycobacterial Catalase-Peroxidase in the Diagnosis and Treatment of Sarcoidosis

(75) Inventors: David Robert Moller, Ellicott City, MD (US); Robert James Cotter, Baltimore, MD (US); Ying Zhang, Ellicott City, MD (US)

Correspondence Address: Foley Hoag, LLP (w/JHV), World Trade Center West, 155 Seaport Blvd, Boston, MA, 02210-2600, US

(73) Assignee: The Johns Hopkins University, Baltimore, MD (US)

(21) Appl. No.: 12/245,476

(22) Filed: Oct. 3, 2008

Related U.S. Application Data
(63) Continuation-in-part of application No. PCT/US07/65860, filed on Apr. 3, 2007.
(60) Provisional application No. 60/788,859, filed on Apr. 3, 2006.

(51) Int. Cl. A61B 10/00; C12Q 1/30; C12Q 1/28; A61K 38/44; A61P 37/00

(52) U.S. Cl. 424/9.81; 435/27; 435/28; 424/94.4

(57) ABSTRACT

Provided are diagnostic and therapeutic methods of treating sarcoidosis comprising the use of microbial catalase-peroxidase protein, its peptide fragments, or derivatives thereof.

### CROSS-REFERENCE TO RELATED APPLICATION

This application is a continuation-in-part application of PCT/US2007/065860, filed Apr. 3, 2007, which claims the benefit of priority to U.S. Provisional Application 60/788,859, filed Apr. 3, 2006. Each of these applications is hereby incorporated herein by reference in its entirety.

### BACKGROUND

Sarcoidosis is a multisystem granulomatous disorder that can result in end-stage fibrosis, cor pulmonale and death. With an approximate incidence in the U.S. of 11-40 per 100,000 people, sarcoidosis represents a significant health problem. The pathologic hallmark of sarcoidosis is noncaseating granulomatous inflammation, which is associated with mononuclear cell infiltration, giant cells and immune complex formation. In general, the initiation of a granulomatous immune response involves an attempt by tissue macrophages to ingest and degrade inciting particulate, antigenic material. This process results in macrophage activation with production of monokines and chemokines. Peptides derived from processed antigenic proteins are displayed in association with class II MHC molecules on the cell surface of dendritic cells and macrophages, stimulating antigen-specific CD4+ T cells. Cytokines and chemokines produced by inflammatory cells and local tissues orchestrate the granulomatous response.

Current evidence supports the concept that granulomatous inflammation in sarcoidosis involves a polarized Th1 immune response to pathogenic tissue antigens. Sites of granulomatous inflammation contain activated CD4+ T cells and mononuclear phagocytes that produce the Th1 cytokines, IFNγ and IL2, and the critical Th1 immunoregulatory monokines, IL12 and IL18. TNF, IL15 and other cytokines contribute to cell activation and granuloma formation.

### SUMMARY

Currently, there is no diagnostic test for sarcoidosis, other than a biopsy showing characteristic granulomas (nodules) in affected tissues when other competing diagnoses are excluded. Further, current treatments for sarcoidosis involve potent anti-inflammatory or immunosuppressive therapies that have significant potential toxicities and are not curative. None are approved by the FDA.

We have discovered that specific microbial proteins in sarcoidosis tissues, mycobacterial catalase-peroxidase protein, are a target of the immune system of patients with sarcoidosis. Accordingly, provided are diagnostic and therapeutic methods of treating sarcoidosis comprising the use of mycobacterial catalase-peroxidase protein, its peptide fragments, or derivatives or variants thereof.

The methods may be incorporated into any test format or device suitable for the practice of the methods. Also provided are kits, reagents, etc. for the practice of the methods.

Further objectives and advantages of the present invention will become apparent as the description proceeds. To gain a full appreciation of the scope of the present invention, it will be further recognized that various aspects of the present invention can be combined to make desirable embodiments of the invention.

### BRIEF DESCRIPTION OF THE DRAWINGS

FIG. 1 depicts protein immunoblots of TX100 extracts of different sarcoidosis and control LN probed with pooled control F(ab′)2 (A), sarcoidosis F(ab′)2 (B), control IgG (C) or sarcoidosis IgG (D) detected with anti-human Fab or Fc linked to horseradish peroxidase using the ECL system (Amersham). Samples were treated with or without proteinase K (PK). Arrows demark antigenic bands seen only in PK-treated sarcoidosis tissues, detected with sarcoidosis but not control F(ab′)2 or IgG.

FIG. 2 depicts protein immunoblot analysis of different fractions of sarkosyl extracts of spleen (A) or lung (B) from sarcoidosis (S) or control (C) using pooled sarcoid sera and an anti-human Fab reagent. The final Pe fractions were solubilized using 8M urea plus β-mercaptoethanol and precipitated before analysis by SDS-PAGE. Arrows demark antigenic bands seen in the Pe fraction from sarcoidosis spleen and lung but not control spleen (control lung without detectable protein in Pe fraction). P=pellet; S=supernatant.

FIG. 3 depicts a table of the peptides matching mKatG peptides shown by m/z from a tryptic digest of a 60-65 kD gel slice from sarcoidosis spleen.

FIG. 4 depicts protein immunoblot analysis of recombinant mKatG (A), sarcoidosis spleen (S11) or LN (LN2) compared with control spleen (C4) or control LN (C2) using IT-57 mAb that binds to M. tuberculosis KatG.

FIG. 5 depicts a protein immunoblot of mKatG and sonicated, cell-free extracts from mycobacterial and bacterial organisms using the anti-mKatG IT57 mAb. IT57 mAbs bind to both the expected ˜80 kD monomer and ˜160 kD homodimer of KatG (31, 32) from Mtb and M. smegmatis extracts but not to extracts from M. chelonei or non-mycobacterial species (10 μg/lane, all extracts). IT57 mAbs also bind to ˜120, 60, 56 and 40 kD peptides derived from recombinant mKatG (lane 2; 100 ng/lane).

FIG. 6 depicts the cellular localization of Mtb katG or 16S rRNA DNA by in situ hybridization. Shown are representative photomicrographs from biopsies of Mtb infected tissue, normal controls (no pathology) and sarcoidosis tissues. Tissue sections were hybridized with or without DIG-labeled probes followed by tyramide signal amplication (mkatG) as previously described. Mtb infected tissue and sarcoidosis samples demonstrated focal collections of Mtb katG and 16S rRNA DNA that were not seen in Wegener granulomatosis, nor in non-granulomatous control tissues. All tissues were negative using reverse probes.

FIG. 7 depicts protein immunoblots with recombinant mKatG (A) or mHsp65 (B) were analyzed with IgG purified from sera of healthy PPD-negative (N), PPD-positive (P) subjects or sarcoidosis (S). Arrowheads demark migration of MW markers (top to bottom: 188, 62, 49, 38 kD). Arrows demark ˜80 and ˜60 kD antigenic bands reactive to mKatG or a 65 kD band reactive to mHsp65.

FIG. 8 depicts the frequency of IFNγ-producing blood (PBMC) T cells from sarcoidosis patients, PPD− or PPD+ healthy controls or BAL from sarcoidosis or non-sarcoidosis lung disease controls (Baltimore) stimulated with mKatG or PPD antigen. PBMC or BAL cells were cultured in vitro with appropriate antigen and IFNγ producing clones measured by ELISPOT. Horizontal bar represents mean+2 SD above control (media alone) condition (for PBMC=10; BAL=15). Positive mKatG specific responses were defined as 2× media control and >10 spots/0.5×10⁶ cells (PBMC) or 2× media control and >15 spots/2×10⁵ cells (BAL).

FIG. 9 depicts representative photomicrographs of lung sections (H&E, bar=50 μm) that were obtained from Lewis rats receiving uncoated control (A) beads or mKatG coated-beads (B, C, D). Significant granulomatous inflammation induced by mKatG coated beads peaks at ˜4d (B) and is still present at 21d (C, D).

FIG. 10 depicts the measuring radius of granulomatous inflammation along orthogonal axes in a rodent model of using mKatG.

FIG. 11 depicts granuloma size in experimental granulomatous inflammation in rats induced by mKatG. Mean values for each group is noted by a bar. Quartiles are indicated by box plots and whiskers (A). Statistically significant comparisons are noted by brackets (*, p<0.05).

FIG. 12 illustrates that mKatG induces antigen-specific Th1 cytokine response in cultured rat splenocytes. Splenocytes were isolated from rats immunized against mKatG (or HDM control) and then exposed to either uncoated or mKatG (or HDM) coated beads. Splenocytes were incubated for 24 h in the presence of media alone, concanavalin A (ConA), recombinant human albumin (Alb), mKatG or HDM. Splenocytes express significantly higher levels of IFNγ when stimulated with mKatG compared with Alb. Values expressed as mean±SE. Statistically significant comparisons are noted by brackets (*, p<0.05).

FIG. 13 depicts representative photomicrographs of lung sections (H&E, bar=50 μm) from C57BL/6J mice 4 days following IV tail vein injection of uncoated beads (A), mKatG-beads (B, D) or HDM-beads (C). Surprisingly, granulomatous inflammation was still observable at 10d (D) with mKatG -beads.

FIG. 14 depicts the proliferation of circulating T cells in response to mKatG in sarcoidosis and healthy controls using ³H-thymidine incorporation assay.

FIG. 15 depicts T cell responses to mKatG in sarcoidosis patients. Frequency of IFNγ-producing PBMC from sarcoidosis and PPD-control subjects in response to mKatG is assessed by ELISPOT.

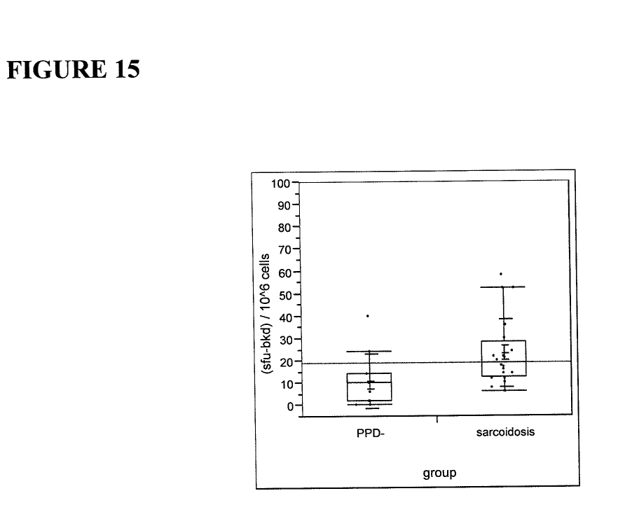

FIG. 16 shows that proliferative responses to mKatG are overall greater in CD3/CD4 compared to CD3/CD8 T cells in the blood and lung of sarcoidosis patients using CFSE staining and flow cytometric methods.

FIG. 17 shows a summary of the proliferative responses of CD4 and CD8 T cells in response to mKatG in sarcoidosis patients.

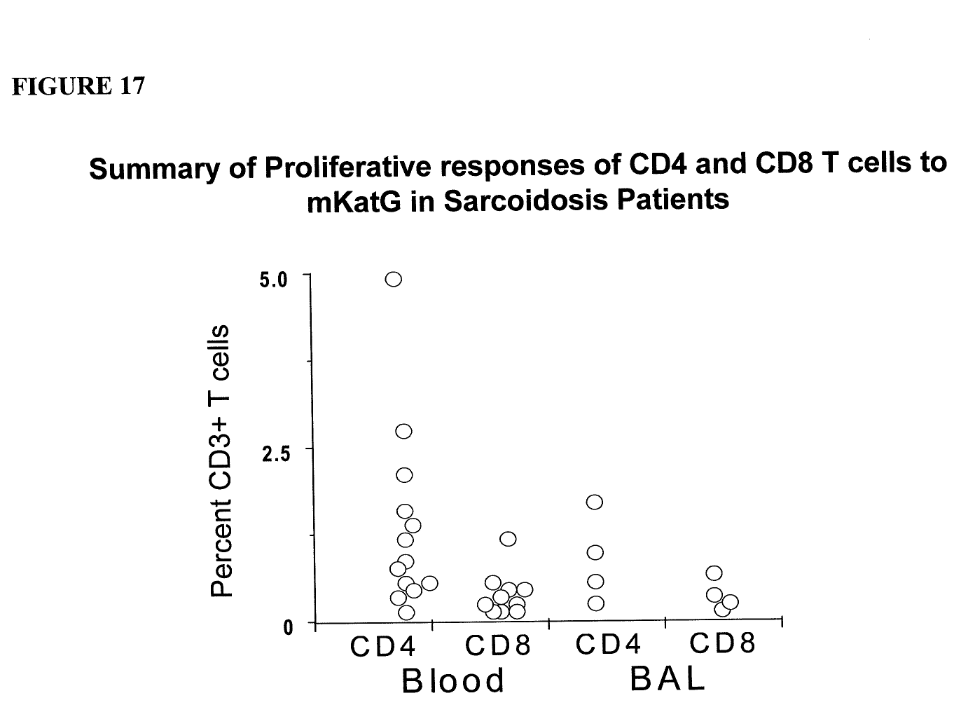

FIG. 18 shows that sarcoidosis BAL T cells from CD3/CD4 and CD3/CD8 subsets (not shown)) produce IFNγ in response to mKatG. In this patient, 5.5% of CD3/CD4 BAL cells produced IFNγ following 6 day incubation with mKatG.

FIG. 19 shows ex vivo T cell reactivity to mKatG in blood from patients with active untreated sarcoidosis and healthy PPD− or PPD+ subjects. PBMC were stimulated with recombinant mKatG and the number of INFγ-producing cells was measured by ELISPOT. A, Dot plots represent mean number of INFγ-producing cells in mKatG wells minus negative control wells per 10⁶ PBMC from individual subjects from the U.S.; group medians indicated by horizontal lines. B, Graph showing proportion of “positive” mKatG responders, defined by samples with a frequency of mKatG-reactive T cells greater than two times media control wells and the absolute number of mKatG-reactive T cells (minus background) exceeding the upper 95% confidence interval level of mKatG responses from PPD− subjects (>13 sfc/10⁶ PBMC).

FIG. 20 shows higher T cell reactivity to mKatG in the lung than blood from patients with sarcoidosis. Bronchoalveolar lavage (BAL) cells or PBMC were stimulated with mKatG and the number of INFγ-producing cells was measured by ELISPOT. Dot plots represent mean number of INFγ-producing cells in mKatG wells minus negative control wells per 10⁶ total BAL cells or PBMC from individual subjects; group medians indicated by horizontal lines.

FIG. 21 shows IFNγ-expressing mKatG-reactive lung and blood T cells derived from both CD4 and CD8 subsets in sarcoidosis. PBMC or BAL cells were incubated for 6 hours with medium alone, mKatG, PPD or SEB, and then intracellularly stained with anti-IFNγ mAb or isotype control. Dot plots represent % cells expressing INFγ within electronically-gated populations of CD3+ CD4+ and CD3+ CD8+T cells. A, Graph represents % IFNγ+T cells in response to mKatG minus media control for PBMC or BAL cells within CD3+ CD4+ and CD3+ CD8+ subsets. B, Graph represents relative proportion of CD4+ or CD8+ T cells expressing INFγ in response to mKatG minus media control calculated as a proportion of total CD3+ T cells reactive to mKatG. Horizontal lines depict median % IFNγ+T cells reactive to mKatG.

FIG. 22 shows effect of treatment, disease inactivity, and chronicity of disease on PBMC reactivity to mKatG. PBMC or BAL cells were stimulated with mKatG and the number of INFγ producing PBMC cells was measured by ELISPOT. A, Dot plots represent mean mKatG reactive PBMC in patients grouped into active untreated vs. active treated vs. inactive sarcoidosis. B, Dot plots represent mean mKatG responses grouped by time since diagnosis in years with medians as indicated.

### DETAILED DESCRIPTION

Unless defined otherwise above, all technical and scientific terms used herein have the same meaning as commonly understood by one of ordinary skill in the art to which this invention belongs. Where a term is provided in the singular, the inventor also contemplates the plural of that term. The nomenclature used herein and the procedures described below are those well known and commonly employed in the art.

The singular forms “a”, “an”, and “the” include plural references unless the context clearly dictates otherwise.

The term “administering” an agent, e.g., a therapeutic agent, or a pharmaceutical composition to a subject or a cell, includes any method of delivery of the same to a subject or a cell, including but not limited to, a subject's system or to a particular region in or on a subject. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof, amino acid analogs having variant side chains; and all stereoisomers of any of any of the foregoing. The names of the natural amino acids are abbreviated herein in accordance with the recommendations of IUPAC-IUB.

The term “antibody” as used herein is intended to include whole antibodies, e.g., of any isotype (IgG, IgA, IgM, IgE, etc), including polyclonal, monoclonal, recombinant and humanized antibodies and fragments thereof which specifically recognize and are able to bind an epitope of a protein. Antibodies can be fragmented using conventional techniques and the fragments screened for utility in the same manner. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFvs may be covalently or non-covalently linked to form antibodies having two or more binding sites.

The term “antigenic portion” refers to a polypeptide fragment or region of a polypeptide that is able to elicit an immune response. An “immune response” refers to the reaction of a subject to the presence of an antigen, which may include at least one of the following: making antibodies, developing immunity, developing hypersensitivity to the antigen, and developing tolerance.

The terms “comprise” and “comprising” is used in the inclusive, open sense, meaning that additional elements may be included.

“Derivative” refers to the chemical modification of a polypeptide sequence, or a polynucleotide sequence. Chemical modifications of a polynucleotide sequence may include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

The term “including” is used herein to mean “including but not limited to”. “Including” and “including but not limited to” are used interchangeably.

The term “microbial catalase or peroxidase protein” refers to any catalase-peroxidase, catalase or peroxidase protein from a microbe, for example, catalase-peroxidase, catalase or peroxidase proteins from mycobacterial species such as Mycobacterium tuberculosis and Mycobacterium smegmatis, or other bacterial species such as Helicobacter pylori and Proprionibacterium acnes.

The terms “polypeptide fragment” or “fragment,” when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

A “patient” or “subject” or “host” refers to either a human or non-human animal.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

A “pharmaceutically-acceptable salt” refers to the relatively non-toxic, inorganic and organic acid addition salts of compounds.

The term “preventing” as used herein is intended to encompass precluding at least one symptom of any condition or disease from occurring, i.e. before it manifests itself in a subject.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis and mass-spectrometry analysis.

The term “Purified Protein Derivative (PPD)+” refers to the Mantoux skin test for tuberculosis, which standardly consists of an intradermal injection of exactly one tenth of a milliliter (mL) of PPD tuberculin.

“Recombinant protein”, “heterologous protein” and “exogenous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

The term “regulatory sequence” is a generic term used throughout the specification to refer to polynucleotide sequences, such as initiation signals, enhancers, regulators and promoters, that are necessary or desirable to affect the expression of coding and non-coding sequences to which they are operably linked. Exemplary regulatory sequences are described in Goeddel; Gene Expression Technology: Methods in Enzymology, Academic Press, San Diego, Calif. (1990), and include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. The nature and use of such control sequences may differ depending upon the host organism. In prokaryotes, such regulatory sequences generally include promoter, ribosomal binding site, and transcription termination sequences. The term “regulatory sequence” is intended to include, at a minimum, components whose presence may influence expression, and may also include additional components whose presence is advantageous, for example, leader sequences and fusion partner sequences. In certain embodiments, transcription of a polynucleotide sequence is under the control of a promoter sequence (or other regulatory sequence) which controls the expression of the polynucleotide in a cell-type in which expression is intended. It will also be understood that the polynucleotide can be under the control of regulatory sequences which are the same or different from those sequences which control expression of the naturally-occurring form of the polynucleotide.

The term “sarcoidosis” (also called sarcoid or Besnier-Boeck disease) as used herein refers to an immune system disorder characterised by non-necrotising granulomas (small inflammatory nodules), as well as related or associated disorders such as celiac disease.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of sequence from a desired sequence (e.g., SEQ. ID NO: 1) that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity

The term “specifically hybridizes” refers to detectable and specific nucleic acid binding. Polynucleotides, oligonucleotides and nucleic acids of the invention selectively hybridize to nucleic acid strands under hybridization and wash conditions that minimize appreciable amounts of detectable binding to nonspecific nucleic acids. Stringent conditions may be used to achieve selective hybridization conditions as known in the art and discussed herein. Generally, the nucleic acid sequence homology between the polynucleotides, oligonucleotides, and nucleic acids of the invention and a nucleic acid sequence of interest will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

As applied to proteins, the term “substantial identity” means that two protein sequences, when optimally aligned, such as by the programs GAP or BESTFIT using default gap weights, typically share at least about 70 percent sequence identity, alternatively at least about 80, 85, 90, 95 percent sequence identity or more. In certain instances, residue positions that are not identical differ by conservative amino acid substitutions.

The term “therapeutically effective amount” refers to that amount of a compound, material, or composition which is sufficient to treat or prevent a disease or disorder. A therapeutically effective amount of a compound can be administered in one or more administrations.

The term “tolerizing effective amount” refers to that amount of a compound, material, or composition which is sufficient to induce tolerance to that compound, material, or composition. A tolerizing effective amount of a compound can be administered in one or more administrations.

The term “treating” as used herein is intended to encompass curing as well as ameliorating at least one symptom of any condition or disease.

The term “vaccine” refers to a substance that elicits an immune response and also confers protective immunity upon a subject.

“Variant,” when used in the context of a polynucleotide sequence, may encompass a polynucleotide sequence related to that of gene X or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

“Vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the invention is intended to include such other forms of expression vectors which serve equivalent functions and which become subsequently known in the art.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

### 1. Microbial Catalase or Peroxidase Protein Compositions

Using techniques described in the Examples below, we identified specific microbial proteins in sarcoidosis tissues, mycobacterial catalase-peroxidase proteins, that are targets of the immune system of patients with sarcoidosis. Thus, provided are isolated recombinant and/or purified microbial catalase or peroxidase polypeptides.

In one embodiment, the polypeptide comprises a sequence having at least about 90%, about 95%, about 96%, about 97%, about 98%, about 99% or about 100% sequence homology to the sequence of Mycobacterium tuberculosis KatG (SEQ ID NO: 1), or to a fragment thereof, e.g., an antigenic fragment:

| | |
|---|---|
| 1 | mpeqhppite tttgaasngc pvvghmkypv egggnqdwwp nrlnlkvlhq npavadpmga |
| 61 | afdyaaevat idvdaltrdi eevmttsqpw wpadyghygp lfirmawhaa gtyrihdgrg |
| 121 | gagggmqrfa plnswpdnas ldkarrllwp vkkkygkkls wadlivfagn calesmgfkt |
| 181 | fgfgfgrvdq wepdevywgk eatwlgdery sgkrdlenpl aavqmgliyv npegpngnpd |
| 241 | pmaaavdire tfrrmamndv etaalivggh tfgkthgagp adlvgpepea apleqmglgw |
| 301 | kssygtgtgk daittgievv wtntptkwdn sfleilygye weltkspaga wqytakdgag |
| 361 | agtipdpfgg pgrsptmlat dlslrvdpiy eritrrwleh peeladefak awyklihrdm |
| 421 | gpvarylgpl vpkqtllwqd pvpavshdlv geaeiaslks qirasgltvs qlvstawaaa |
| 481 | ssfrgsdkrg ganggrirlq pqvgwevndp dgdlrkvirt leeiqesfns aapgnikvsf |
| 541 | adlvvlggca aiekaakaag hnitvpftpg rtdasqeqtd vesfavlepk adgfrnylgk |
| 601 | gnplpaeyml ldkanlltls apemtvlvgg lrvlganykr lplgvfteas esltndffvn |
| 661 | lldmgitwep spaddgtyqg kdgsrkvkwt gsrvdlvfgs nselralvev ygaddaqpkf |
| 721 | vqdfvaawdk vmnldrfdvr |

In another embodiment, the polypeptide comprises a sequence having at least about 90%, about 95%, about 96%, about 97%, about 98%, about 99% or about 100% sequence homology to the sequence of Mycobacterium smegmatis KatG (SEQ ID NO: 2), or to a fragment thereof, e.g., an antigenic fragment:

| | |
|---|---|
| 1 | mpedrpieds ppigeaqtda paggcpagfg rikppvaggs nxdwwpnqln mkilqknpdv |
| 61 | inpldedfdy rsavqnldvd airadivevm htsqdwwpad fghygplfir mawhaagtyr |
| 121 | vsdgrggaga gmqrfaplns wpdnasldka rrllwpvkkk ygknlswadl ivyagnvale |
| 181 | dmgfrtagfa fgredrwepe edvywgpeqe wldrtkrytg erdlenplaa vqmgliyvnp |
| 241 | egpngnpdpq asaidiretf grmamndvet aalivgghtf gkthgngdas lvgpepeaap |
| 301 | leevglgwrn pqgtgvgkda itsglevtwt htptkwdnsf leilygnewe ltkspaganq |
| 361 | wkpkdngwan svplahedgk thpsmltsdl alrvdpiyeq itrrwldhpe elaeefakaw |
| 421 | fkllhrdmgp vtrylgpevp kdtwlwqdni pagndlsdde vaklkeliad sgltvsqlvs |
| 481 | tawkaastfr ssdlrggang grirlqpqlg weanepdela qvvrkyeeiq kasginvsfa |
| 541 | dlvvlggnvg vekaakaagf dvtvpftpgr gdatqeetdv dsfaylepka dgfrnylgkg |
| 601 | sdlpaefkli dranllglsa pemttlvggl rvldvnhggt khgvltdkpg alttdffvnl |
| 661 | ldmstawkps paddgtyigt dratgspkwt gtrvdlvfas nsqlralaev yaeddskekf |
| 721 | vkdfvaawtk vmdadrfdva |

In another embodiment, the polypeptide comprises a sequence having at least about 90%, about 95%, about 96%, about 97%, about 98%, about 99% or about 100% sequence homology to the sequence of Proprionibacterium acnes KPA171202 catalase (SEQ ID NO: 3), or to a fragment thereof, e.g., an antigenic fragment:

| | |
|---|---|
| 1 | mpidpskpst ntngspapsd eysltvgadg pvvlhdahli dtlahfnren iperkphakg |
| 61 | sgafghlevt advseytkas flqkgretpm larfstvage lgspdtwrdv rgfslkfytd |
| 121 | egnfdmvgnn tpvffmrdpm kfphfirsqk rlpnsglrsp nmmydywsls pesahqvayl |
| 181 | mgprgiplsy rtmngyssht yswvnaegki twvkyhfisd qgvhnmtadr akaivgdhpd |
| 241 | ihredlfnhi adgdypswtv kvqlmpydea kdyrfnpfdi tkvwphkdyp lhtigkftld |
| 301 | rnpenffaqi eqaafspsnt vegtglspdr mllgrvfayn daarnrlgvn yeqlpvnrpt |
| 361 | tptnqytfdg qmafehsgsa ptyapnsygr dfatgyrtgs eatweadgel vrsaqtrhae |
| 421 | dddfgqahal vhdvfsdaer delvdtlvdl mtnfdmeepv ignalsywrn idagiaeate |
| 481 | gri |

In yet another embodiment, the polypeptide comprises a sequence having at least about 90%, about 95%, about 96%, about 97%, about 98%, about 99% or about 100% sequence homology to the sequence of Proprionibacterium acnes KPA171202 thiol peroxidase (SEQ ID NO: 4), or to a fragment thereof, e.g., an antigenic fragment:

| | |
|---|---|
| 1 | mattafmgkp lntvgdlpqv gsllpsftlv ksdlselrsd elkgkklvln ifpsvdtgvc |
| 61 | atsvrtfnek aaglddttvl cvsrdlpfaq arfcgaegik nvvvasafrs hfgkdlgvtl |
| 121 | adgpmqhlla raivvvdaeg kvtytqlvde ittepdydaa leaaska |

In still another embodiment, the polypeptide comprises a sequence having at least about 90%, about 95%, about 96%, about 97%, about 98%, about 99% or about 100% sequence homology to the sequence of Helicobacter pylori J99 catalase (SEQ ID NO: 5), or to a fragment thereof, e.g., an antigenic fragment:

| | |
|---|---|
| 1 | mvnkdvkqtt afgapvwddn nvitagprgp vllqstwfle klaafdreri pervvhakgs |
| 61 | gaygtftvtk ditkytkaki fskvgkktec ffrfstvage kgsadavrdp rgfamkyyte |
| 121 | egnwdlvgnn tpvffirdai kfpdfihtqk rdpqtnlpnp dmvwdfwsnv peslyqvtwv |
| 181 | msdrgipksf rhmdgfgsht fslinakger fwvkfhfetm qgvkhltnee aaevrkydpd |
| 241 | snqrdlfdai aggdfpkwkm siqympeeda kkyrfhpfdv tkiwylqdyp lmevgiveln |
| 301 | knpenyfaev eqaaftpanv vpgigyspdr mlqgrlfsyg dthryrlgvn ypqipvnrpr |
| 361 | cpfhsssrdg ymqngyygsl qnytpsslpg ykedksardp kfnlahieke fevwnwdyra |
| 421 | edsdyytqpg dyyrslpade kerlydtigg slahvthkei vdkqlehfkk adpkyaegvk |
| 481 | kalekhqkmm kdmhakdmhh mkkkk |

In certain embodiments, the subject polypeptides may comprise a fusion protein of any of the above-described polypeptides containing at least one domain which increases its solubility and/or facilitates its purification, identification, detection, and/or delivery. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, a polypeptide of the invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. Linker sequences between a polypeptide of the invention and the fusion domain may be included in order to facilitate construction of the fusion protein or to optimize protein expression or structural constraints of the fusion protein. In another embodiment, the polypeptide may be constructed so as to contain protease cleavage sites between the fusion polypeptide and polypeptide of the invention in order to remove the tag after protein expression or thereafter. Examples of suitable endoproteases, include, for example, Factor Xa and TEV proteases.

In another embodiment, the subject polypeptides may be modified so that the rate of traversing the cellular membrane is increased. For example, the polypeptide may be fused to a second peptide which promotes “transcytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, polypeptides may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally-occurring membrane-translocating sequence (MTS), such as the peptide sequences disclosed in U.S. Pat. No. 6,248,558.

In another embodiment, truncated polypeptides may be prepared. Truncated polypeptides have from 1 to 20 or more amino acid residues removed from either or both the N- and C-termini. Such truncated polypeptides may prove more amenable to expression, purification or characterization than the full-length polypeptide. In addition, the use of truncated polypeptides may also identify stable and active domains of the full-length polypeptide that may be more amenable to characterization or incorporation into a pharmaceutical composition.

It is also possible to modify the structure of the polypeptides of the invention for such purposes as enhancing therapeutic or prophylactic efficacy, or stability (e.g., ex vivo shelf life, resistance to proteolytic degradation in vivo, etc.). Such modified polypeptides, when designed to retain at least one activity of the naturally-occurring form of the protein, are considered “functional equivalents” of the polypeptides described in more detail herein. Such modified polypeptides may be produced, for instance, by amino acid substitution, deletion, or addition, which substitutions may consist in whole or part by conservative amino acid substitutions. For example, substitutions, deletions or additions of about 1, 2, 3, 4, 5, 6, 7, 8, 9, 10, 15, 20 or more amino acids are contemplated.

For instance, it is reasonable to expect that an isolated conservative amino acid substitution, such as replacement of a leucine with an isoleucine or valine, an aspartate with a glutamate, a threonine with a serine, will not have a major effect on the biological activity of the resulting molecule. Whether a change in the amino acid sequence of a polypeptide results in a functional homolog may be readily determined by assessing the ability of the variant polypeptide to produce a response similar to that of the wild-type protein. Polypeptides in which more than one replacement has taken place may readily be tested in the same manner.

Protein homologs may be generated combinatorially. In a representative embodiment of this method, the amino acid sequences for a population of protein homologs are aligned, preferably to promote the highest homology possible. Such a population of variants may include, for example, homologs from one or more species, or homologs from the same species but which differ due to mutation. Amino acids which appear at each position of the aligned sequences are selected to create a degenerate set of combinatorial sequences. In certain embodiments, the combinatorial library is produced by way of a degenerate library of genes encoding a library of polypeptides which each include at least a portion of potential protein sequences. For instance, a mixture of synthetic oligonucleotides may be enzymatically ligated into gene sequences such that the degenerate set of potential nucleotide sequences are expressible as individual polypeptides, or alternatively, as a set of larger fusion proteins (e.g. for phage display).

There are many ways by which the library of potential homologs may be generated from a degenerate oligonucleotide sequence. Chemical synthesis of a degenerate gene sequence may be carried out in an automatic DNA synthesizer, and the synthetic genes may then be ligated into an appropriate vector for expression. One purpose of a degenerate set of genes is to provide, in one mixture, all of the sequences encoding the desired set of potential protein sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp. 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis may be utilized to generate a combinatorial library. For example, protein homologs may be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated forms of proteins that are bioactive.

Another aspect of the invention relates to polypeptide fragments derived from the full-length polypeptides of the invention, e.g., about 5, 6, 7, 8, 9, 10, 15, 20, 25, 30, 35, 40, 50 or more contiguous amino acids from SEQ ID NOs: 1 through 5, and/or antigenic fragments thereof, as described further herein. Fragments of the polypeptides may be produced using standard polypeptide synthesis methods as will be known to one of skill in the art. Alternatively, such polypeptide fragments, as well as the subject polypeptides, may be produced using recombinant techniques. Chemical synthesis of polypeptides of the invention may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Schnolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

Provided also are isolated nucleic acid sequences that encode all or a substantial portion of the amino acid sequences set forth in SEQ ID NOs: 1 through 5 or other polypeptides described above, as well as vectors, host cells, and cultures for the expression and production thereof or for gene therapy methods. Also provided are nucleic acids comprising a nucleotide sequence that is at least about 80%, 90%, 95%, 98% or 99% identical to any of SEQ ID NOs 1 through 5 or a portion thereof and may encode a homolog of a catalase or peroxidase.

For example, the coding sequence for SEQ ID NO: 1 is SEQ ID NO: 6:

| | |
|---|---|
| 1 | gtgcccgagc aacacccacc cattacagaa accaccaccg gagccgctag caacggctgt |
| 61 | cccgtcgtgg gtcatatgaa ataccccgtc gagggcggcg gaaaccagga ctggtggccc |
| 121 | aaccggctca atctgaaggt actgcaccaa aacccggccg tcgctgaccc gatgggtgcg |
| 181 | gcgttcgact atgccgcgga ggtcgcgacc atcgacgttg acgccctgac gcgggacatc |
| 241 | gaggaagtga tgaccacctc gcagccgtgg tggcccgccg actacggcca ctacgggccg |
| 301 | ctgtttatcc ggatggcgtg gcacgctgcc ggcacctacc gcatccacga cggccgcggc |
| 361 | ggcgccgggg gcggcatgca gcggttcgcg ccgcttaaca gctggcccga caacgccagc |
| 421 | ttggacaagg cgcgccggct gctgtggccg gtcaagaaga agtacggcaa gaagctctca |
| 481 | tgggcggacc tgattgtttt cgccggcaac tgcgcgctgg aatcgatggg cttcaagacg |
| 541 | ttcgggttcg gcttcggccg ggtcgaccag tgggagcccg atgaggtcta ttggggcaag |
| 601 | gaagccacct ggctcggcga tgagcgttac agcggtaagc gggatctgga gaacccgctg |
| 661 | gccgcggtgc agatggggct gatctacgtg aacccggagg ggccgaacgg caacccggac |
| 721 | cccatggccg cggcggtcga cattcgcgag acgtttcggc gcatggccat gaacgacgtc |
| 781 | gaaacagcgg cgctgatcgt cggcggtcac actttcggta agacccatgg cgccggcccg |
| 841 | gccgatctgg tcggccccga acccgaggct gctccgctgg agcagatggg cttgggctgg |
| 901 | aagagctcgt atggcaccgg aaccggtaag gacgcgatca ccaccggcat cgaggtcgta |
| 961 | tggacgaaca ccccgacgaa atgggacaac agtttcctcg agatcctgta cggctacgag |
| 1021 | tgggagctga cgaagagccc tgctggcgct tggcaataca ccgccaagga cggcgccggt |
| 1081 | gccggcacca tcccggaccc gttcggcggg ccagggcgct ccccgacgat gctggccact |
| 1141 | gacctctcgc tgcgggtgga tccgatctat gagcggatca cgcgtcgctg gctggaacac |
| 1201 | cccgaggaat tggccgacga gttcgccaag gcctggtaca agctgatcca ccgagacatg |
| 1261 | ggtcccgttg cgagatacct tgggccgctg gtccccaagc agaccctgct gtggcaggat |
| 1321 | ccggtccctg cggtcagcca cgacctcgtc ggcgaagccg agattgccag ccttaagagc |
| 1381 | cagatccggg catcgggatt gactgtctca cagctagttt cgaccgcatg ggcggcggcg |
| 1441 | tcgtcgttcc gtggtagcga caagcgcggc ggcgccaacg gtggtcgcat ccgcctgcag |
| 1501 | ccacaagtcg ggtgggaggt caacgacccc gacggggatc tgcgcaaggt cattcgcacc |
| 1561 | ctggaagaga tccaggagtc attcaactcc gcggcgccgg ggaacatcaa agtgtccttc |
| 1621 | gccgacctcg tcgtgctcgg tggctgtgcc gccatagaga aagcagcaaa ggcggctggc |
| 1681 | cacaacatca cggtgccctt caccccgggc cgcacggatg cgtcgcagga acaaaccgac |
| 1741 | gtggaatcct ttgccgtgct ggagcccaag gcagatggct tccgaaacta cctcggaaag |
| 1801 | ggcaacccgt tgccggccga gtacatgctg ctcgacaagg cgaacctgct tacgctcagt |
| 1861 | gcccctgaga tgacggtgct ggtaggtggc ctgcgcgtcc tcggcgcaaa ctacaagcgc |
| 1921 | ttaccgctgg gcgtgttcac cgaggcctcc gagtcactga ccaacgactt cttcgtgaac |
| 1981 | ctgctcgaca tgggtatcac ctgggagccc tcgccagcag atgacgggac ctaccagggc |
| 2041 | aaggatggca gtcgcaaggt gaagtggacc ggcagccgcg tggacctggt cttcgggtcc |
| 2101 | aactcggagt tgcgggcgct tgtcgaggtc tatggcgccg atgacgcgca gccgaagttc |
| 2161 | gtgcaggact tcgtcgctgc ctgggacaag gtgatgaacc tcgacaggtt cgacgtgcgc |
| 2221 | tga |

Further, the coding sequence for SEQ ID NO: 2 is SEQ ID NO: 7:

| | |
|---|---|
| 1 | atgcctgagg atcgcccgat cgaagacagc ccgcccatcg gagaagctca gaccgacgcg |
| 61 | cctgcgggtg gttgcccggc gggtttcggc cgcatcaagc ctccggtggc cggtggcagc |
| 121 | aacsgtgact ggtggcccaa tcagctcaat atgaagatcc tgcagaagaa cccggacgtc |
| 181 | atcaatccgc tggacgagga tttcgactac cggtccgcgg ttcagaacct cgatgtcgac |
| 241 | gcgctgcgcg ccgacatcgt cgaggtcatg cacacgtcgc aggactggtg gcctgccgac |
| 301 | ttcggccact acggaccgct gttcatccgc atggcctggc acgccgcggg cacctaccgc |
| 361 | gtcagtgacg gccgcggcgg tgcgggcgcg ggcatgcagc gcttcgcgcc gctgaacagc |
| 421 | tggcccgaca acgcgagcct ggacaaggcc cgccgcctgc tgtggccggt caagaagaag |
| 481 | tacggcaaga acctgtcgtg ggccgacctg atcgtgtatg ccggcaatgt ggcgctggag |
| 541 | gacatgggct tccgcaccgc cggcttcgcg ttcggtcgcg aggaccgctg ggagcccgag |
| 601 | gaggacgtgt actggggtcc cgagcaggaa tggctcgacc gaacaaagcg ctacaccggt |
| 661 | gagcgtgacc tggagaaccc gcttgccgcg gtccagatgg gcctcatcta cgtcaacccc |
| 721 | gaaggcccca acggcaatcc ggatccgcag gcctcggcca tcgacatccg cgagacgttc |
| 781 | ggccggatgg cgatgaacga cgtcgagacc gccgcgctga tcgtcggtgg ccacaccttc |
| 841 | ggcaagacgc acggcaacgg cgacgcctcg ctggtgggcc ccgagccgga agccgccccg |
| 901 | ctcgaagagg tcggcctcgg ctggcgcaac ccgcagggca ccggcgtcgg caaggacgcc |
| 961 | atcaccagcg gtctcgaggt cacctggacc cacacgccga ccaagtggga caacagcttc |
| 1021 | ctggagatcc tgtacggcaa cgagtgggag ctcaccaaga gccccgcggg cgccaaccag |
| 1081 | tggaagccca aggacaacgg ctgggccaac tcggtgccgc tggcccacga ggacggcaag |
| 1141 | acccacccgt cgatgctgac ctcggatctc gcgctgcgcg tcgacccgat ctacgagcag |
| 1201 | atcacccgtc gctggctgga tcaccccgag gaacttgccg aggagttcgc caaggcgtgg |
| 1261 | ttcaagctgc tgcaccgcga catgggtccg gtgacccgct acctgggccc cgaggtgccg |
| 1321 | aaggacacct ggctgtggca ggacaacatc ccggcgggca acgatctgtc cgacgacgag |
| 1381 | gtggccaagc tcaaggagct gatcgccgac tcgggtctga ctgtgtcgca gctggtttcg |
| 1441 | accgcgtgga aggccgcgtc gaccttccgg tcgagtgacc tgcgcggcgg cgccaacggc |
| 1501 | ggccgcatcc gcctgcagcc gcagctgggc tgggaggcca acgagcccga cgagctcgcc |
| 1561 | caggtggtcc gcaagtacga ggagatccag aaggcgtccg gcatcaacgt gtcgttcgcc |
| 1621 | gacctggtgg tcctcggcgg caacgtgggt gtcgagaagg ccgccaaggc agccggattc |
| 1681 | gacgtcaccg tgccgttcac gccgggccgt ggcgacgcca cccaggaaga gaccgacgtc |
| 1741 | gactcgttcg cctacctgga gcccaaggcc gacggcttcc gcaactacct gggcaagggt |
| 1801 | tcggacctgc ccgcggagtt caagctgatc gaccgggcga acctgctggg cctgtcggct |
| 1861 | cctgagatga ccacgctcgt cggcggtctg cgggtgctcg acgtcaacca cggcggtacc |
| 1921 | aagcacggcg tgctgaccga caagccgggc gcgttgacca cggacttctt cgtcaacctg |
| 1981 | ctcgacatgt ccaccgcatg gaagccctcg cctgccgatg acggcaccta catcggcacc |
| 2041 | gaccgtgcca ccggttcgcc caagtggacc ggcacgcgtg tcgatctggt gttcgcgtcg |
| 2101 | aactcgcagc tgcgggccct ggccgaggtg tacgcggagg atgactccaa ggagaagttc |
| 2161 | gtcaaggact tcgtcgccgc gtggaccaag gtgatggacg ccgaccgctt cgacgtcgcc |
| 2221 | tga |

Further, the coding sequence for SEQ ID NO: 3 is SEQ ID NO: 8:

| | |
|---|---|
| 1 | atgcctattg atcccagcaa gccgtcaacg aacaccaacg ggtcgccagc gcctagcgac |
| 61 | gaatactccc tgaccgtcgg cgctgacggc ccggtagtgc tccacgacgc acatctcatc |
| 121 | gacaccctgg cccatttcaa ccgggaaaac atccccgaac gcaagcctca cgccaaggga |
| 181 | tcgggggcct tcggacatct cgaggttacc gctgacgtct cggagtacac caaggcatcc |
| 241 | ttcctgcaga aggggcgcga aacccccatg ctggcccgat tctccacagt cgccggtgag |
| 301 | ctcggcagcc ctgacacctg gcgtgatgtg cgtggattct cgttgaagtt ctacaccgac |
| 361 | gagggcaact tcgacatggt cggcaacaac accccggtgt tcttcatgcg tgacccgatg |
| 421 | aagttcccgc atttcatccg ctcccagaaa cggttaccga attccggact gcgcagtccg |
| 481 | aatatgatgt atgactattg gagcctgtcc cccgaatcgg cccatcaagt ggcttacctc |
| 541 | atgggaccgc gcggcattcc gctcagctat cgcactatga acggctactc ctcgcacacc |
| 601 | tactcatggg ttaacgccga agggaagatc acctgggtga aataccactt catcagtgac |
| 661 | cagggtgttc acaacatgac tgctgaccgg gccaaagcga tcgtcggtga tcacccagac |
| 721 | atccaccgcg aggacctctt caaccacatt gccgacggcg actatccgtc ctggaccgtc |
| 781 | aaggtgcagc tcatgcccta tgacgaagcc aaggattacc ggttcaaccc gttcgacatc |
| 841 | accaaggtgt ggccgcataa ggattacccg ctgcacacca tcggcaagtt cactctggac |
| 901 | cgcaacccgg agaacttctt cgcccagatc gagcaagcgg ccttctcccc gtcgaacacc |
| 961 | gttgagggca ctgggttgtc cccagaccgg atgctgctgg gtcgtgtctt cgcctacaac |
| 1021 | gatgccgccc gcaaccgtct gggggtcaac tatgaacaac tccctgtgaa ccggccaacg |
| 1081 | acgccgacaa accagtacac cttcgacggc cagatggcct tcgaacactc cggttcggca |
| 1141 | cccacctacg ccccgaactc ctacggtcgc gattttgcca ccggataccg aaccggttcc |
| 1201 | gaagctacct gggaggctga cggcgaactc gttcgttccg cccagacccg gcacgcggaa |
| 1261 | gatgacgact tcggtcaggc ccacgccctg gtgcatgacg tcttttccga cgcagagcgt |
| 1321 | gacgagctcg tcgatacttt ggtggacttg atgaccaatt tcgacatgga agagccggtc |
| 1381 | atcggcaacg cgctgtccta ctggcgcaac atcgacgccg gtattgccga ggcaaccgag |
| 1441 | ggcagaatct ga |

Further, the coding sequence for SEQ ID NO: 4 is SEQ ID NO: 9:

| | |
|---|---|
| 1 | atggctacta ccgctttcat gggaaaaccc cttaacaccg tcggggacct tcctcaggtc |
| 61 | ggttccttgc tgccgtcgtt caccctcgtc aagagtgact tgtcagagct gagaagcgat |
| 121 | gagttgaagg gcaagaagct cgtcctcaac attttcccaa gtgtggacac cggggtgtgt |
| 181 | gcaacgagtg tgcgcacctt caacgagaaa gccgctggac tcgacgacac caccgtctta |
| 241 | tgtgtttctc gtgaccttcc attcgcccag gctcgcttct gcggtgcgga gggcatcaag |
| 301 | aacgttgttg ttgcctcagc cttccgctcc cattttggta aggaccttgg cgttaccctc |
| 361 | gccgatggcc cgatgcagca tctgcttgcg cgcgccatcg tcgtcgtgga cgctgaggga |
| 421 | aaggttacct acacccagct cgttgatgag atcactaccg agccggatta cgacgctgcc |
| 481 | cttgaggctg cgtccaaggc ctga |

Further, the coding sequence for SEQ ID NO: 5 is SEQ ID NO: 10:

| | |
|---|---|
| 1 | mvnkdvkqtt afgapvwddn nvitagprgp vllqstwfle klaafdreri pervvhakgs |
| 61 | gaygtftvtk ditkytkaki fskvgkktec ffrfstvage kgsadavrdp rgfamkyyte |
| 121 | egnwdlvgnn tpvffirdai kfpdfihtqk rdpqtnlpnp dmvwdfwsnv peslyqvtwv |
| 181 | msdrgipksf rhmdgfgsht fslinakger fwvkfhfetm qgvkhltnee aaevrkydpd |
| 241 | snqrdlfdai aggdfpkwkm siqympeeda kkyrfhpfdv tkiwylqdyp imevgiveln |
| 301 | knpenyfaev eqaaftpanv vpgigyspdr mlqgrlfsyg dthryrlgvn ypqipvnrpr |
| 361 | cpfhsssrdg ymqngyygsl qnytpsslpg ykedksardp kfnlahieke fevwnwdyra |
| 421 | edsdyytqpg dyyrslpade kerlydtigg slahvthkei vdkqlehfkk adpkyaegvk |
| 481 | kalekhqkmm kdmhakdmhh mkkkk |

Isolated nucleic acids which differ from the subject nucleic acids due to degeneracy in the genetic code are also provided. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the polypeptides will exist. One skilled in the art will appreciate that these variations in one or more nucleotides (from less than 1% up to about 3 or 5% or possibly more of the nucleotides) of the nucleic acids encoding a particular protein of the invention may exist among a given species due to natural allelic variation. Any and all such nucleotide variations and resulting amino acid polymorphisms are also provided.

Bias in codon choice within genes in a single species appears related to the level of expression of the protein encoded by that gene. Accordingly, further provided are nucleic acid sequences which have been optimized for improved expression in a host cell by altering the frequency of codon usage in the nucleic acid sequence to approach the frequency of preferred codon usage of the host cell. Due to codon degeneracy, it is possible to optimize the nucleotide sequence without affecting the amino acid sequence of an encoded polypeptide.

Nucleic acids may also contain linker sequences, modified restriction endonuclease sites and other sequences useful for molecular cloning, expression or purification of such recombinant polypeptides.

The subject nucleic acids may be used to cause expression and over-expression of a subject polypeptide in cells propagated in culture, e.g. to produce proteins or polypeptides, including fusion proteins or polypeptides.

Provided are host cells transfected with a recombinant gene in order to express a subject polypeptide. The host cell may be any prokaryotic or eukaryotic cell. For example, a polypeptide may be expressed in bacterial cells, such as E. coli, insect cells (baculovirus), yeast, or mammalian cells. In those instances when the host cell is human, it may or may not be in a live subject. Other suitable host cells are known to those skilled in the art. Additionally, the host cell may be supplemented with tRNA molecules not typically found in the host so as to optimize expression of the polypeptide. Other methods suitable for maximizing expression of the polypeptide will be known to those in the art.

The present invention further pertains to methods of producing the polypeptides. For example, a host cell transfected with an expression vector encoding a polypeptide may be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated.

A cell culture includes host cells, media and other byproducts. Suitable media for cell culture are well known in the art. The polypeptide may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of a polypeptide.

Thus, a nucleotide sequence encoding all or a selected portion of polypeptide of the invention, may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant polypeptides of the invention by microbial means or tissue-culture technology.

Expression vehicles for production of a recombinant protein include plasmids and other vectors. For instance, suitable vectors for the expression of a polypeptide of the invention include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83). These vectors may replicate in E. coli due the presence of the pBR322 ori, and in S. cerevisiae due to the replication determinant of the yeast 2 micron plasmid. In addition, drug resistance markers such as ampicillin may be used.

In certain embodiments, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant protein by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In another variation, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. In vitro transcription and translation may be coupled in a one-pot reaction to produce proteins from one or more isolated DNAs.

When expression of a carboxy terminal fragment of a polypeptide is desired, i.e. a truncation mutant, it may be necessary to add a start codon (ATG) to the oligonucleotide fragment containing the desired sequence to be expressed. It is well known in the art that a methionine at the N-terminal position may be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, may be achieved either in vivo by expressing such recombinant polypeptides in a host which produces MAP (e.g., E. coli or CM89 or S. cerevisiae), or in vitro by use of purified MAP (e.g., procedure of Miller et al.).

Coding sequences for a polypeptide of interest may be incorporated as a part of a fusion gene including a nucleotide sequence encoding a different polypeptide. Further provided is an isolated nucleic acid comprising a subject nucleic acid and at least one heterologous sequence encoding a heterologous peptide linked in frame to the nucleotide sequence of the nucleic acid so as to encode a fusion protein comprising the heterologous polypeptide. The heterologous polypeptide may be fused to (a) the C-terminus of the polypeptide encoded by the nucleic acid, (b) the N-terminus of the polypeptide, or (c) the C-terminus and the N-terminus of the polypeptide. In certain instances, the heterologous sequence encodes a polypeptide permitting the detection, isolation, solubilization and/or stabilization of the polypeptide to which it is fused. In still other embodiments, the heterologous sequence encodes a polypeptide selected from the group consisting of a polyHis tag, myc, HA, GST, protein A, protein G, calmodulin-binding peptide, thioredoxin, maltose-binding protein, poly arginine, poly His-Asp, FLAG, a portion of an immunoglobulin protein, and a transcytosis peptide.

Fusion expression systems can be useful when it is desirable to produce an immunogenic fragment of a polypeptide of the invention. For example, the VP6 capsid protein of rotavirus may be used as an immunologic carrier protein for portions of polypeptide, either in the monomeric form or in the form of a viral particle. The nucleic acid sequences corresponding to the portion of a polypeptide of the invention to which antibodies are to be raised may be incorporated into a fusion gene construct which includes coding sequences for a late vaccinia virus structural protein to produce a set of recombinant viruses expressing fusion proteins comprising a portion of the protein as part of the virion. The Hepatitis B surface antigen may also be utilized in this role as well. Similarly, chimeric constructs coding for fusion proteins containing a portion of a polypeptide of the invention and the poliovirus capsid protein may be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992).

### 2. Diagnostic Methods

Further provided are in vitro and in vivo diagnostic methods comprising the use of at least one of microbial catalase or peroxidase proteins described above.

The diagnostic tests contemplated for sarcoidosis are based on testing immune reactivities to a range of microbial catalases or peroxidases. Diagnostic assays provided herein include measuring antigen-specific T and B cell responses to specific microbial catalases or peroxidases as determined from T cell assays (including but not limited to skin testing, ELISPOT and flow cytometry) and B cell assays (including but not limited to protein immunoblot or ELISA assays). Other diagnostic assays provided herein including using microbial catalases or peroxidases in skin test-delayed type hypersensitivity (purified protein derivative PPD equivalent) assays, skin test-delayed granuloma reaction (Kveim equivalent) assays and in vitro tests (protein immunoblot or T cell assays). Based on the results of the immunologic profile of T and B cell responses to specific microbial catalases or peroxidases, a specific vaccine based therapy for individual patients with sarcoidosis may be developed based on the specific immune reactivities of individual sarcoidosis patients. Vaccine based therapies, described in detail further below, may comprise the use of recombinant proteins, peptides or peptide mimetics derived from those specific microbial catalases or peroxidases to which the patient responds in laboratory or skin testing.

A profile of immune responses to microbial catalases or peroxidases that includes measures of both T cell responsiveness (determined from assays including but not limited to ELISPOT and flow cytometry or skin testing) and the presence or absence of B cell responses (determined from assays including but not limited to protein immunoblot or ELISA assays) may also serve as a prognostic tool to predict the likelihood of the subsequent clinical course of sarcoidosis, for example, whether the course of sarcoidosis will be one of remission or will follow a course of chronic persistent or progressive disease. Further included is using a profile of immune responses to microbial catalases or peroxidases derived from mycobacterial and non-mycobacterial microbes as a test of disease “activity” (the presence and degree of underlying granulomatous inflammation), the need for treatment, the effectiveness of treatment (using standard therapies including but not limited to corticosteroids, immunosuppressive and anti-TNF therapies) and as an aid for treatment selection. Such a test may be employed at multiple times during the clinical course of sarcoidosis. This test may be used together with other patient information derived from tests including but not limited to genetic tests, proteomic profiles of tissues or blood, or other tests of general immunity in sarcoidosis patients, in order to enhance the test characteristics as a diagnostic tool or as an aid in clinical management.

In one embodiment, a method for determining whether a subject has or is likely to develop sarcoidosis may comprise: (a) administering to the skin of a subject a microbial catalase or peroxidase protein or antigenic portion thereof, and (b) determining the presence of a reaction to the microbial catalase or peroxidase or antigenic portion thereof, wherein the presence of a reaction indicates that the subject has or is likely to develop sarcoidosis.

In certain embodiments, the reaction is an immune reaction, immediate hypersensitivity reaction, a delayed type hypersensitivity reaction or the development or presence of a granulomatous nodule. The presence of one or more reactions may be determined in the method. Methods for determining reactions such as an immune reaction, immediate hypersensitivity reaction, a delayed type hypersensitivity reaction or the development or presence of a granulomatous nodule are well-known in the art.

In certain embodiments, the determination of the presence of an immediate hypersensitivity reaction may be conducted about 5 to 30 minutes after administering a microbial catalase or peroxidase protein or antigenic portion thereof to the skin.

In other embodiments, the determination of the presence of a delayed type hypersensitivity reaction may be conducted about 48 to 72 hours after administering a microbial catalase or peroxidase protein or antigenic portion thereof to the skin.

In still other embodiments, the determination of the presence of a granulomatous nodule is conducted about 2 to 4 weeks after contacting the skin with a microbial catalase or peroxidase protein or antigenic portion thereof.

The microbial catalase or peroxidase protein or antigenic portion thereof may be administered to the skin of the subject by any method of administration suitable for dermal administration, for example, intradermal injection.

In another embodiment, a method for determining whether a subject has or is likely to develop sarcoidosis may comprise determining whether the subject has an immune or skin test reaction to a microbial catalase or peroxidase protein or portion thereof, wherein the presence of a microbial catalase or peroxidase protein or portion thereof and the fact that the subject is not a subject who has or has had tuberculosis or is Purified Protein Derivative (PPD)+, indicates that the subject has or is likely to develop sarcoidosis.

In still another embodiment, a method for determining whether a subject has or is likely to develop sarcoidosis, comprising determining whether the subject has immune cells or antibodies reactive to a microbial catalase or peroxidase protein, wherein the presence of immune cells reactive to a microbial catalase or peroxidase protein and the fact that the subject is not a subject who has or has had tuberculosis or is Purified Protein Derivative (PPD)+, indicates that the subject has or is likely to develop sarcoidosis. The method may further comprise obtaining a sample from the subject and determining the presence of the immune cells or antibodies in the sample. The sample may be, for example, a blood sample, bronchoalveolar lavage fluid from a bronchoscopy or similar procedure, a sputum sample, etc. Determining the presence of immune cells reactive to a microbial catalase or peroxidase protein may also comprise contacting immune cells of the subject with a microbial catalase or peroxidase protein or an antigenic portion thereof. It may further comprise determining antigen specific T cell responses, for example, by flow cytometry.

An assay may comprise using 1, 2, 3, 4, 5, or more microbial catalases or peroxidases or fragments thereof. For example, a skin test may comprise administering to the skin a catalase or peroxidase from M. tuberculosis, from M. smegmatis, from H. pylori, from P. acnes, and/or one or more of any other mycobacterial or non-mycobacterial microorganisms, or portions of one or more of these proteins.

An assay may also comprise using another antigen from a mycobacterial or non-mycobacterial microorganism, e.g., ESAT-6 (Drake, et al., Infection Immunity 75:527 (2007) or hsp65.

An assay may also comprise determining the presence in a subject of one or more antibodies that bind specifically to a sarcoidosis-associated antigen, e.g., mKatG, ESAT-6, hsp65, etc. Assays may also comprise a combination of one or more assays set forth herein.

Certain assays may comprise determining the presence of an antigen or antigen-specific B cell, T cell or antibody wherein the antigen is a mycobacterial or non-mycobacterial catalase or peroxidase, with the proviso that the antigen is not M. tuberculosis or M. smegmatis catalase mKatG.

The presence of antigens or antigen-specific B or T cells may be determined in any tissue or bodily fluid, e.g., lung, spleen, blood, etc.

The above-described diagnostic methods and assays may be used to diagnose sarcoidosis or other diseases similar to or related to sarcoidosis that implicate the same antigens, for example, tuberculosis, leprosy or other non-tuberculous mycobacterial infections, Blau's syndrome (very similar disease to sarcoidosis, though Kveim negative; familial), Crohn's disease in which there is some evidence for a role for mycobacterial organisms in the disease (or other microbial catalases or peroxidases), chronic beryllium disease, and other autoimmune diseases, particularly granulomatous disorders such as Wegener's granulomatosis, rheumatoid arthritis, granulomatous hepatitis, granulomatous uveitis.

### 3. Vaccine Compositions and Methods of Use

As discussed above, based on the results of the immunologic profile of T and B cell responses to specific microbial catalases or peroxidases, vaccine-based therapies for individual patients with sarcoidosis may be developed based on the specific immune reactivities of individual sarcoidosis patients.

Vaccine based therapies may comprise the use of recombinant proteins, peptides or peptide mimetics derived from those specific microbial catalases or peroxidases to which the patient responds in laboratory or skin testing. Accordingly, provided also are vaccine compositions comprising at least one microbial catalase or peroxidase.

The vaccine-based therapies may also be used in treating and preventing tuberculosis, leprosy or other non-tuberculous mycobacterial infections, Blau's syndrome (very similar disease to sarcoidosis, though Kveim negative; familial), Crohn's disease in which there is some evidence for a role for mycobacterial organisms in the disease (or other microbial catalases or peroxidases), chronic beryllium disease, and other autoimmune diseases, particularly granulomatous disorders such as Wegener's granulomatosis, rheumatoid arthritis, granulomatous hepatitis, granulomatous uveitis.

In one embodiment, a method for treating or preventing sarcoidosis in a subject may comprise administering to a subject in need thereof a tolerizing effective amount of a microbial catalase or peroxidase protein or antigenic portion thereof, such that sarcoidosis is treated or prevented in a subject. The tolerizing effective amount of a microbial catalase or peroxidase protein or antigenic portion thereof may be administered at about regular intervals for about six months to 3 years.

In another embodiment, a method for treating sarcoidosis in a subject may comprise administering to a subject in need thereof a therapeutically effective amount of a microbial catalase or peroxidase protein or antigenic portion thereof, to induce an immune reaction against the protein, such that sarcoidosis is treated or prevented in a subject.

Dosage levels of between about 0.0001 milligrams (0.1 microgram) and about 2.5 mg/kg body weight, preferably between about 0.05 and about 0.5 mg/kg body weight, and most preferably between about 0.10 and about 0.23 mg/kg body weight are useful in the methods described herein, and in particular for dosages of an oral vaccine. A subcutaneous vaccine may in certain embodiments use lower dosages, i.e., about 0.0001 microgram to about 10 micrograms. The amount of active ingredient that may be combined with the carrier materials to produce a single dosage form will vary depending upon the host treated and the particular mode of administration. The dose of the microbial catalase or peroxidase protein or antigenic portion thereof may vary according to factors such as the disease state, age, sex, and weight of the individual, and the ability of antibody to elicit a desired response in the individual. Dosage regime may be adjusted to provide the optimum therapeutic response. For example, several divided doses may be administered daily or the dose may be proportionally reduced as indicated by the exigencies of the therapeutic situation. The dose of the vaccine may also be varied to provide optimum preventative dose response depending upon the circumstances.

The microbial catalase or peroxidase protein or antigenic portion thereof may in certain embodiments be administered orally to the subject, such as to induce oral tolerance to the protein.

Further provided are pharmaceutical or vaccine compositions comprising a tolerizing and/or therapeutically effective amount of a microbial catalase or peroxidase protein or antigenic portion thereof and a pharmaceutically acceptable vehicle.

The compositions are suitable for administration to subjects in a biologically compatible form in vivo. The expression “biologically compatible form suitable for administration in vivo” as used herein means a form of the substance to be administered in which any toxic effects are outweighed by the therapeutic effects. The substances may be administered to any animal, preferably humans.

The compositions of the present invention may additionally contain suitable diluents, adjuvants and/or carriers. Preferably, the compositions contain an adjuvant which can enhance the immunogenicity of the vaccine in vivo. The adjuvant may be selected from many known adjuvants in the art including the lipid-A portion of gram negative bacteria endotoxin, trehalose dimycolate of mycobacteria, the phospholipid lysolecithin, dimethyldictadecyl ammonium bromide (DDA), certain linear polyoxypropylene-polyoxyethylene (POP-POE) block polymers, aluminum hydroxide, liposomes and CpG (cytosine-phosphate-guanidine) polymers. The vaccines may also include cytokines that are known to enhance the immune response including GM-CSF, IL-2, IL-12, TNF and IFNγ.

The compositions of the instant invention may be formulated and introduced as a vaccine through oral, intradermal, intramuscular, intraperitoneal, intravenous, subcutaneous, intranasal, and intravaginal, or any other standard route of immunization.

In formulations of the subject compositions, wetting agents, emulsifiers and lubricants, such as sodium lauryl sulfate and magnesium stearate, as well as coloring agents, releasing agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any method well known in the art of pharmacy. The amount of composition that may be combined with a carrier material to produce a single dose may vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations, which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants, which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

In addition, vaccines may be administered parenterally as injections (intravenous, intramuscular or subcutaneous). The vaccine compositions of the present invention may optionally contain one or more adjuvants. Any suitable adjuvant can be used, such as CpG polymers, aluminum hydroxide, aluminum phosphate, plant and animal oils, and the like, with the amount of adjuvant depending on the nature of the particular adjuvant employed. In addition, the anti-infective vaccine compositions may also contain at least one stabilizer, such as carbohydrates such as sorbitol, mannitol, starch, sucrose, dextrin, and glucose, as well as proteins such as albumin or casein, and buffers such as alkali metal phosphates and the like.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers, which may be employed in the pharmaceutical compositions of the invention, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

### 4. Kits

The present invention provides kits, for example for diagnosis, preventing or treating sarcoidosis. For example, a kit may comprise one or more polypeptides, optionally formulated as pharmaceutical compositions as described above and optionally instructions for their use. In still other embodiments, the invention provides kits comprising one or more one or more polypeptides, optionally formulated as pharmaceutical compositions, and one or more devices for accomplishing administration of such compositions.

Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, therapy, and other applications.

### 5. Additional Embodiments

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims. Although this disclosure has been described in terms of certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure.

For example, in addition to the embodiments disclosed herein, including the claims appended hereto, the following paragraphs set forth additional, non-limiting embodiments (with all references to paragraphs contained in this section referring to other paragraphs set forth in this section):

1. A method for determining whether a subject has or is likely to develop sarcoidosis, comprising

administering to the skin of a subject a microbial catalase or peroxidase protein or antigenic portion thereof, and

determining the presence of a reaction to the microbial catalase or peroxidase or antigenic portion thereof, wherein the presence of a reaction indicates that the subject has or is likely to develop sarcoidosis.

2. The method of paragraph 1, wherein the reaction is an immune reaction.

3. The method of paragraph 2, wherein the immune reaction is an immediate hypersensitivity reaction.

4. The method of paragraph 3, wherein determining the presence of an immediate hypersensitivity reaction is conducted about 5 to 30 minutes after administering a microbial catalase or peroxidase protein or antigenic portion thereof to the skin.

5. The method of paragraph 2, wherein the immune reaction is a delayed type hypersensitivity reaction.

6. The method of paragraph 5, wherein determining the presence of a delayed type hypersensitivity reaction is conducted about 48-72 hours after administering a microbial catalase or peroxidase protein or antigenic portion thereof to the skin.

7. The method of paragraph 1, wherein the reaction is the development of a granulomatous nodule.

8. The method of paragraph 7, wherein determining the presence of a granulomatous nodule is conducted about 2-4 weeks after contacting the skin with a microbial catalase or peroxidase protein or antigenic portion thereof.

9. The method of paragraph 3, further comprising determining the presence of a delayed type hypersensitivity reaction and/or the presence of a granulomatous nodule.

10. The method of paragraph 5, further comprising determining the presence of a granulomatous nodule.

11. The method of paragraph 1, wherein the microbial catalase or peroxidase protein or antigenic portion thereof is administered to the skin of the subject by intradermal injection.

12. The method of paragraph 1, wherein the microbial catalase or peroxidase protein is a mycobacterial catalase or peroxidase protein.

13. The method of paragraph 12, wherein the protein is a Mycobacterium tuberculosis protein.

14. The method of paragraph 13, wherein the protein is catalase-peroxidase KatG.

15. The method of paragraph 1, wherein the microbial catalase or peroxidase protein is a Helicobacter pylori catalase.

16. The method of paragraph 1, wherein the microbial catalase or peroxidase protein is a recombinant protein.

17. A method for determining whether a subject has or is likely to develop sarcoidosis, comprising determining whether the subject has a microbial catalase or peroxidase protein or portion thereof, wherein the presence of a microbial catalase or peroxidase protein or portion thereof and the fact that the subject is not a subject who has or has had tuberculosis or is Purified Protein Derivative (PPD)+, indicates that the subject has or is likely to develop sarcoidosis.

18. A method for determining whether a subject has or is likely to develop sarcoidosis, comprising determining whether the subject has immune cells reactive to a microbial catalase or peroxidase protein, wherein the presence of immune cells reactive to a microbial catalase or peroxidase protein and the fact that the subject is not a subject who has or has had tuberculosis or is Purified Protein Derivative (PPD)+, indicates that the subject has or is likely to develop sarcoidosis.

19. The method of paragraph 18, comprising obtaining a blood sample from the subject and determining the presence of the immune cells in the blood sample.

20. The method of paragraph 19, wherein determining the presence of immune cells reactive to a microbial catalase or peroxidase protein comprises contacting immune cells of the subject with a microbial catalase or peroxidase protein or antigenic portion thereof.

21. The method of paragraph 20, further comprising determining antigen specific T cell responses.

22. The method of paragraph 21, wherein T cell responses are determined by flow cytometry.

23. A kit for diagnosing sarcoidosis comprising at least one composition comprising a microbial catalase or peroxidase protein or antigenic portion thereof, and optionally instructions for use.

24. A method for treating or preventing sarcoidosis in a subject, comprising administering to a subject in need thereof a tolerizing effective amount of a microbial catalase or peroxidase protein or antigenic portion thereof, such that sarcoidosis is treated or prevented in a subject.

25. The method of paragraph 24, wherein the tolerizing effective amount of a microbial catalase or peroxidase protein or antigenic portion thereof is administered at about regular intervals for about six months to 3 years.

26. The method of paragraph 25, wherein the microbial catalase or peroxidase protein or antigenic portion thereof is administered orally to the subject, such as to induce oral tolerance to the protein.

27. A method for treating sarcoidosis in a subject, comprising administering to a subject in need thereof a therapeutically effective amount of a microbial catalase or peroxidase protein or antigenic portion thereof, to induce an immune reaction against the protein, such that sarcoidosis is treated or prevented in a subject.

28. A pharmaceutical composition comprising a tolerizing effective amount of a microbial catalase or peroxidase protein or antigenic portion thereof and a pharmaceutically acceptable vehicle.

29. A pharmaceutical composition comprising a therapeutically effective amount of a microbial catalase or peroxidase protein or antigenic portion thereof and a pharmaceutically acceptable vehicle.

### EXEMPLIFICATION

The invention, having been generally described, may be more readily understood by reference to the following examples, which are included merely for purposes of illustration of certain aspects and embodiments of the present invention, and are not intended to limit the invention in any way. All headings are for the convenience of the reader and should not be used to limit the meaning of the text that follows the heading, unless so specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed. ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### Example 1

### Sarcoidosis is an Antigen-Driven Disorder

Direct evidence that sarcoidosis is an antigen-driven disorder was provided by studies from our laboratory and others showing that sarcoidosis is characterized by the selective expansion of oligoclonal populations of T-cells at sites of disease, some with T cell receptor (TCR) hypervariable region amino acid similarities, consistent with MHC-restricted antigen-driven responses. We identified oligoclonal T cell populations with restricted TCR gene usage in lung (BAL cells), blood and skin (Kveim reaction). Attempts to identity the nature of stimulating antigens by cloning TCR-specific T cells were not initially successful. More recently, using a limited proteomic approach, we have identified a pathogenic antigen, mKatG in sarcoidosis.

### Example 2

### Sarcoidosis is a Polarized Th1 Disorder

Using BAL as a research tool, we confirmed that pulmonary sarcoidosis is associated with upregulated expression of the Th1 cytokines IFNγ and IL2 with little or no Th2 cytokines, IL4 or IL5. We demonstrated that the major immunoregulatory Th1 cytokine, IL12, is upregulated in the sarcoidosis lung, being produced by BAL macrophages both constitutively and in response to bacterial reagents, consistent with a local dysregulated Th1 response in sarcoidosis.

### Example 3

### A Mycobacterial Protein, mKatG is a Pathogenic Antigen in Sarcoidosis

We employed a limited proteomic approach using protein immunoblot and MALDI-TOF mass spectrometry (MS) to identify candidate tissue antigens, followed by patient studies to validate candidate antigens as targets of the immune response in sarcoidosis. The results of these studies identifying mKatG as a candidate antigen have recently been published (Song et al . J Exp Med. (2005); 201:755-67); a brief synopsis is provided below.

### Example 4

### Protease-Resistant Proteins in Sarcoidosis Tissue are Targets of Sarcoidosis IgG

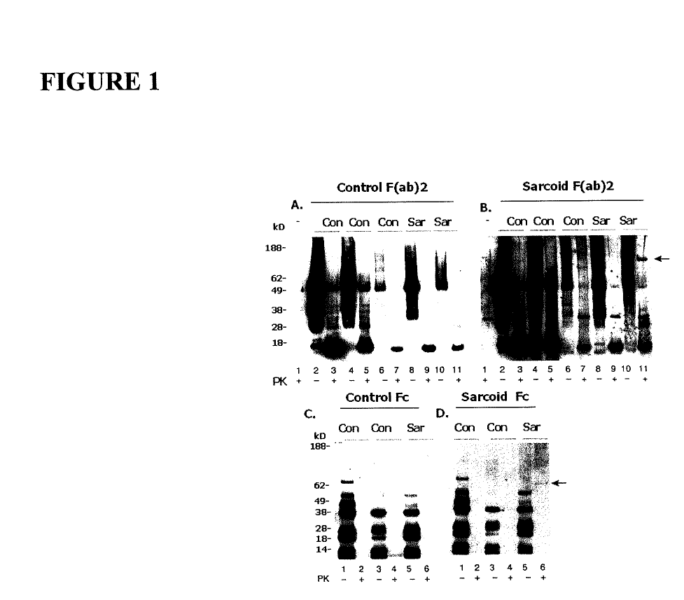

To identify tissue antigens relevant to immune-mediated granulomatous inflammation in sarcoidosis, we prepared detergent extracts of sarcoidosis and control tissues using protocols that previously were reported to preserve and concentrate the in vivo biologic activity of the granuloma-inducing factor in Kveim reagent (FIG. 1). This limited proteomics approach detected TX-100 insoluble, protease-resistant antigens in sarcoidosis tissues that were targets of circulating IgG or F(ab′)₂ fragments from sarcoidosis patients but not control reagents or secondary antibody alone. Overall, antigenic bands of 140-160, 80-90, 62-64 or 60 kD sizes were seen in 9 of 12 (75%) sarcoidosis tissues but only 2 of 17 (12%) control tissues (all 62-64 kD size) (p=0.0013). Thus, protease digestion allows detection of TX100 insoluble tissue antigens in sarcoidosis tissues detected with sarcoidosis IgG but not control IgG consistent with disease relevant antigens.

### Example 4

### Sarkosyl-Extracted Sarcoidosis Tissue Extracts Contain Proteins that are Targets of Sarcoidosis IgG

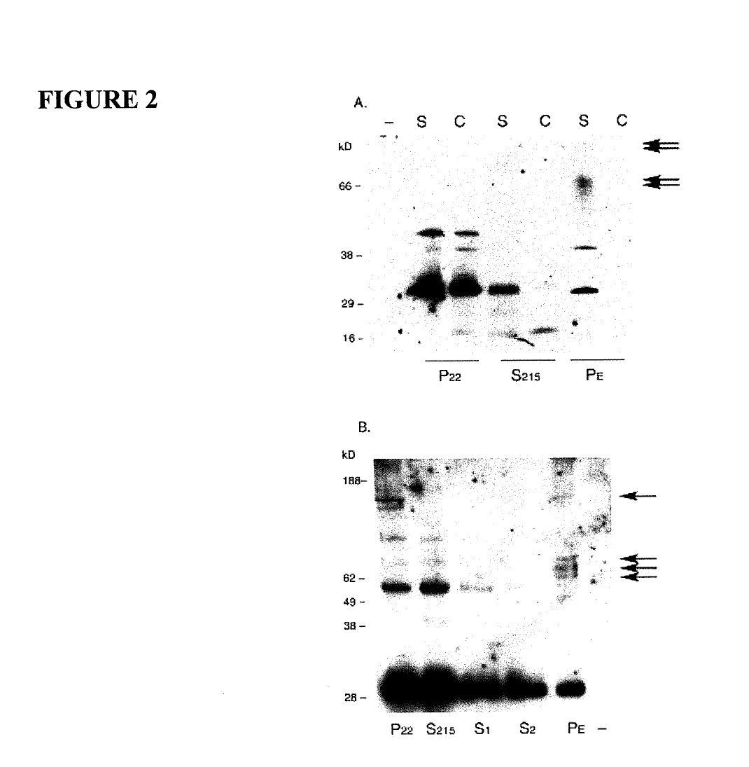

We utilized a sarkosyl extraction protocol used to enrich for protease-resistant prion PrPSc aggregates given the similar biochemical characteristics of the active Kveim component. We found 60, 62, 64 and 140-160 kD bands in the final precipitated (Pe) fraction from a sarcoidosis spleen that was bound by pooled sarcoidosis IgG (FIG. 2). The Pe fraction from 5 of 6 control spleens contained little detectable protein, and did not demonstrate antigenic bands to either sarcoidosis or control IgG reagents. The Pe fraction of a sarcoidosis lung tissue demonstrated several bands including those of approximately 60, 62, 66, 80, and 140-160 kD sizes that were not seen with secondary antibody alone (FIG. 2). These antigens were similar in size to those in TX100 processed tissues, suggesting these tissue antigens are identical. Thus, sarcoidosis tissue antigens are poorly soluble in sarkosyl consistent with the granuloma-inducing agent of Kveim reagent.

### Example 5

### Identification of Candidate Tissue Antigens Using MALDI-TOF Mass Spectroscopy

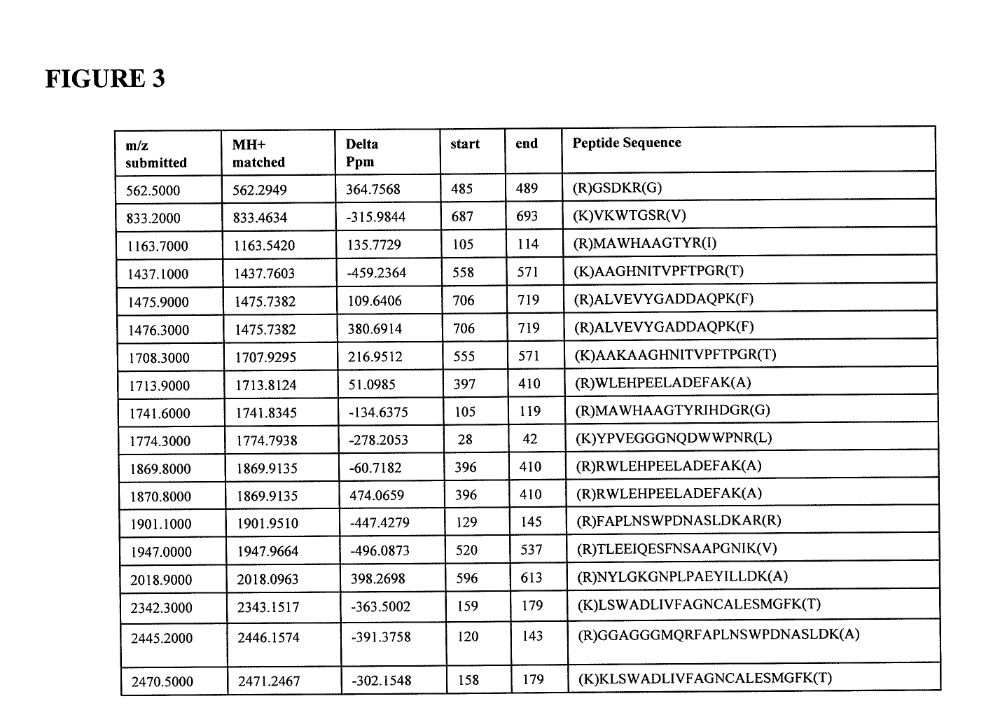

To identify candidate antigenic proteins in sarcoidosis tissues, gel bands were cut, digested with trypsin in-situ and the resulting peptides extracted and analyzed by MALDI-TOF MS. The peptide mass fingerprint was analyzed by protein database sequence searching using Protein Prospector (prospector.ucsfedu) and the Mascot (Matrix Science, www.matrixscience.com) search engines. A 60-62 kD band from sarcoidosis spleen demonstrated a highly significant match using both search engines to M. tuberculosis KatG protein (MW=80625) (MOWSE score 1.07×10e+09 with 16/74 peptide matches (FIG. 3). The mass spectrum of a 60-62 kDa band excised from a sarcoidosis lung had 4/26 peptides that matched M. smegmatis KatG (MOWSE score 1.39×10e03) (FIG. 3). Control tissue gels did not contain sufficient protein for analysis. Thus, KatG proteins are found in the poorly soluble tissue fraction of sarcoidosis tissues.

### Example 6

### Validation of MALDI-TOF MS/Peptide Fingerprinting with Protein Immunoblot Analysis

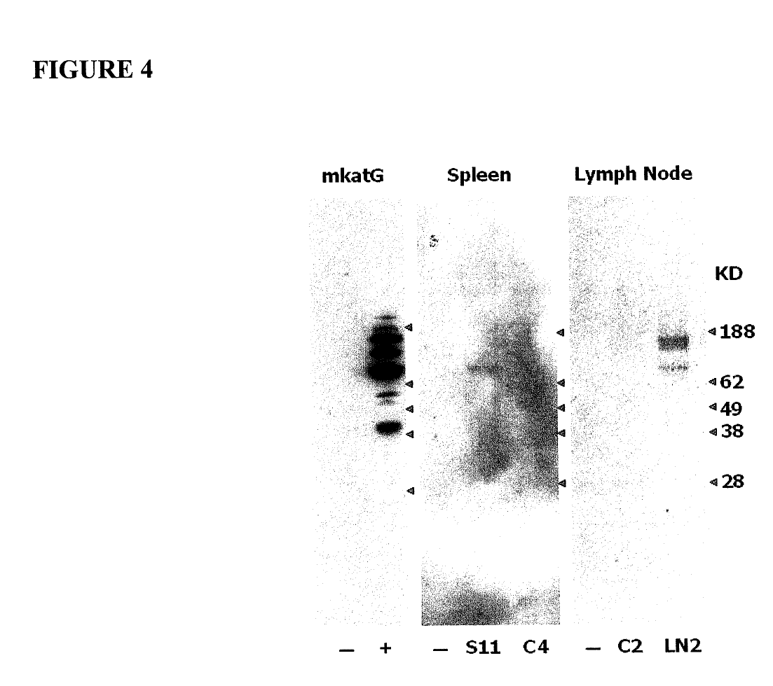

To confirm the presence of mKatG protein in sarcoidosis tissue, protein immunoblots were probed with a mAb reactive to M. tuberculosis KatG protein (IT57, from Colorado State University under NIH, NIAID contract N01 AI-75320). We found that five of 9 (55%) sarcoidosis tissues but 0/14 (0%) control tissues had detectable mKatG protein on immunoblots using anti-mKatG mAbs (p=0.0037) (FIG. 4).

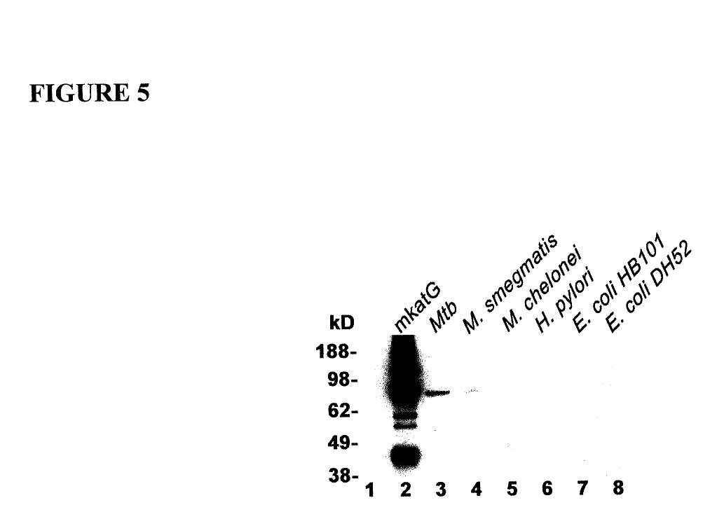

To assess the specificity of the IT57 mAb, we analyzed cell-free extracts from several mycobacterial and non-mycobacterial commensal bacteria. We found the IT57 mAb binds to proteins from Mtb, M. smegmatis but not M. chelonei, nor H. pylori nor E. coli (FIG. 5).

Thus, subsets of sarcoidosis tissues but not control tissues have mKatG protein in poorly soluble tissue extracts using protein immunoblotting and anti-mKatG mAbs that specifically bind mKatG from Mtb and select non-tuberculous mycobacteriae.

### Example 7

### DNA from mkatG and Mycobacterial 16S RNA is Present in Sarcoidosis Tissues

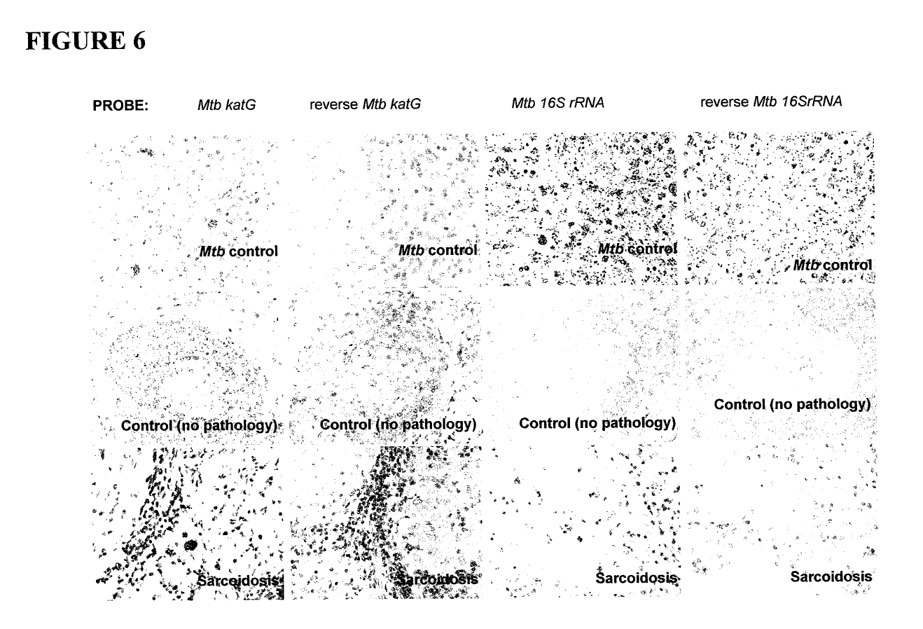

To further validate the presence of mKatG in sarcoidosis tissues, we evaluated whether mkatG DNA and mycobacterial 16S RNA DNA could be detected in sarcoidosis tissues. Using in situ hybridization with tyramide amplification, we detected Mtb katG DNA in 7/18 (39%) sarcoidosis and 4/4 Mtb infected tissues but not in 0/18 control tissues (sarcoidosis vs. non-tuberculous controls, p=0.0076) (FIG. 6). SH without signal amplification employing probes derived from Mtb 16S rRNA confirmed the presence of mycobacterial DNA in 5/6 (83%) mkatG-positive samples and overall in 6/16 (38%) sarcoidosis tissues compared with 0/16 (0%) control tissues (p=0.018). Reverse Mtb 16S rRNA probes were uniformly negative in all samples. A subset of sarcoidosis tissues but not control tissues contain of Mtb katG DNA and/or Mtb 16S RNA DNA.

### Example 8

### mKatG is a Target of the Adaptive Immune Response in Sarcoidosis

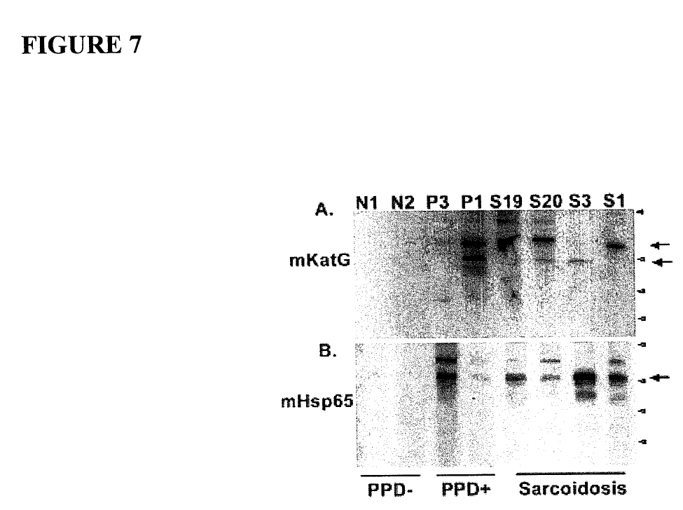

To evaluate whether mKatG was a target of the adaptive immune response in sarcoidosis, the M. tuberculosis katG gene was cloned and the protein expressed, purified and used as an antigen in protein immunoblots. IgG antibodies to recombinant mKatG were detected in 12/25 (48%) sarcoidosis patients compared to 0/11 (0%) PPD− (p=0.0059) and 4/10 (40%) PPD+ (p=0.7233) control subjects (FIG. 7). Remnant mycobacterial catalase/peroxidase is a pathogenic tissue antigen that is the target of the adaptive immune responses driving granulomatous inflammation in a subgroup of sarcoidosis patients.

### Example 9

### mKatG is a Target of the Adaptive T Cell Response in a Majority of Sarcoidosis Patients

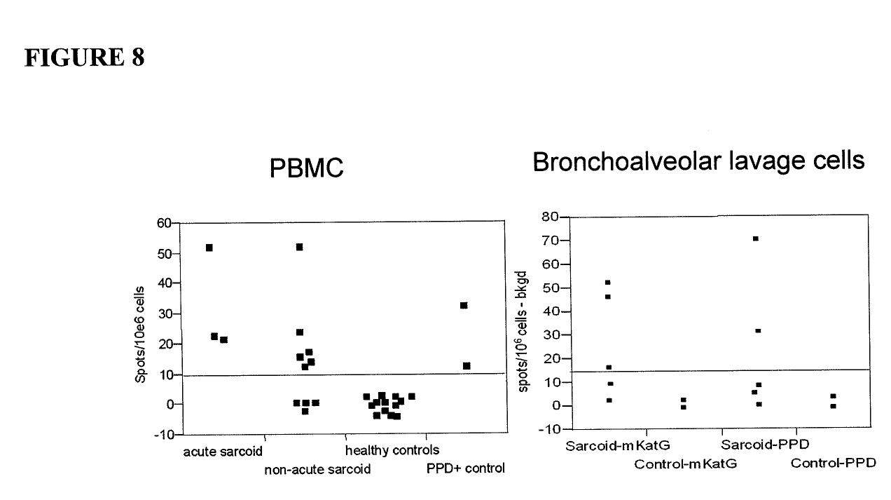

To evaluate antigen specific T cell responses to mKatG in sarcoidosis, we have utilized ELISPOT assays to determine the frequency of mKatG-specific, IFNγ-producing T cells in the blood and lung (BAL) from patients with sarcoidosis and PPD+ and PPD− healthy controls (FIG. 8). In our study, to date we have found significant IFNγ-producing mKatG specific T cell responses in 9/13 (69%) patients, including 3 of 3 (100%) patients with acute sarcoidosis (Lofgren or acute arthritis). Lower responses were seen to PPD and mHsp65 antigens in these patients. Similarly, to date, we have found 3 of 5 (60%) patients undergoing diagnostic bronchoscopy (in whom a diagnosis of sarcoidosis was later confirmed), to have positive mKatG responses by lung T cells. We noted a higher frequency of unstimulated IFNγ-producing T cell clones in BAL compared to blood-consistent with a higher frequency of constitutive IFNγ-producing T cells in the lung.

To begin to dissect the molecular details of the T cell responses to mKatG in sarcoidosis, we performed peptide analysis of the mKatG protein. If mKatG were a dominant pathogenic antigen, we would expect that MHC genes associated with sarcoidosis from genetic studies to bind mKatG peptides and stimulate disease relevant T cell clones. To test this possibility, we analyzed Mtb KatG for peptides predicted to bind to MHC molecules relevant to sarcoidosis using peptide binding prediction algorithms available over the internet (e.g., www.imtech.res.in/raghava/propred/). Given the size of the mKatG protein, we initially selected peptides predicted to bind to MHC DRB1 *0101,*0301,*0410,*1101,1120,*1501,*0701. Given the number of possible stimulatory peptides and limitations in patient blood donations, we have restricted our peptide analysis to 5 peptides at a time plus the recombinant mKatG and control antigens. Altogether, we have been testing 20 mKatG-derived peptides.

To date, we have had positive responses most frequently to the following peptides: P2, P4, P8, P14. Since the 20 peptides have not all been evaluated in the same patients, comparison studies are premature. However, our preliminary data suggest that T cell responses to the recombinant mKatG protein predicts responses to one or more peptides. Interestingly, to date, 3 of the 4 peptides most frequently associated with T cell responses are predicted to bind to HLA DRB1*0301, the same MHC molecule implicated in expansion of Vα2.3 T cells in good prognosis sarcoidosis in Sweden. It will be of interest to dissect the fine specificity of T cell responses in comparison between US and Swedish patients, given the high proportion of Swedish patients with good prognosis sarcoidosis.

### Example 10

### mKatG is a Target of the Adaptive B Cell Response in a Majority of Sarcoidosis Patients

To further explore the prevalence and association with sarcoidosis subgroups, we expanded our original analysis of the IgG response in sarcoidosis patients. Our original study included mostly patients with untreated but established, chronic sarcoidosis—the overall proportion of sarcoidosis patients with positive IgG titers to recombinant mKatG in protein immunoblots was 48% vs. 0% in PPD− healthy controls. Our expanded studies show a higher proportion of patients with mKatG IgG titers in those patients newly diagnosed, and with systemic and pulmonary involvement. To date, these studies of new subjects have shown circulating anti-mKatG IgG in 10/16 (62%) sarcoidosis patients vs. 1/6 new PPD− (or unknown) healthy controls and 2/2 PPD+ healthy controls. These results may be a more accurate reflection of early sarcoidosis. Additional studies seem indicated to define the sensitivity of mKatG IgG responses in patients with newly discovered disease to assess the possibility of an adjunctive diagnostic serologic test in PPD− patients.

Although the molecular details of the T and B cell responses to mKatG in sarcoidosis and tuberculosis remain uncertain, it may be relevant that cytokine expression in sarcoidosis appears to be more polarized towards Th1 cytokine expression than in tuberculosis. This finding alone predicts that quantitative and qualitative differences in the humoral and cellular responses to mycobacterial antigens exist between these two disorders. The persistence of large mycobacterial proteins in tissues may be particularly relevant to long-term immunologic stimulation in sarcoidosis from the slow degradation and release of mycobacterial peptides or from fostering the development of autoimmunity. The possibility that dominant Th1 immune responses may favor a pathogenic humoral response has precedence in murine models of myasthenia gravis. In either case, establishing the sensitivity and specificity of mKatG IgG responses in sarcoidosis subgroups, disease and healthy controls may provide clues to disease immune regulation.

### Example 15

### Experimental Models of mKatG-Induced Granulomatous Inflammation

To investigate the mechanisms of mKatG antigen-driven granulomatous inflammation, and thus develop an animal model of sarcoidosis, we coated Sepharose 4B beads (Amersham Biosciences) with mKatG according to the manufacturer's recommendations, and introduced the beads into animals by intravenous (IV) tail vein injection using Lewis rats. Rats were immunized to mKatG in complete Freund's adjuvant (CFA) prior to mKatG-bead injection.

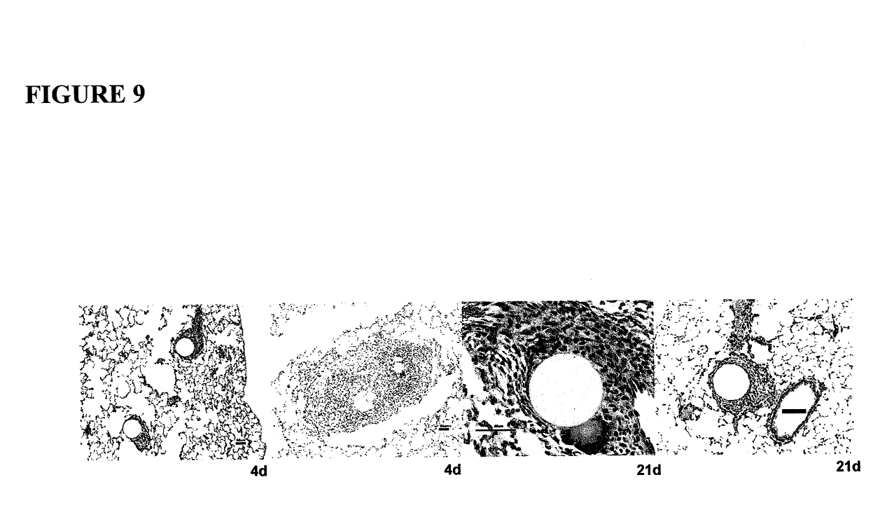

A robust monocyte/macrophage predominant infiltrate with epithelioid appearing cells developed 4 days following mKatG coated bead infusion (FIG. 9 ). This mononuclear cell infiltration was diminished but still present at 21 days (FIGS. 9 C and 9 D), a feature not found in previous reports of murine models of granulomatous inflammation. Chronic granulomatous inflammation appeared to be associated with the development of fibrosis as suggested by increased matrix staining in a portion of the 21 day granulomata (FIG. 9 D).

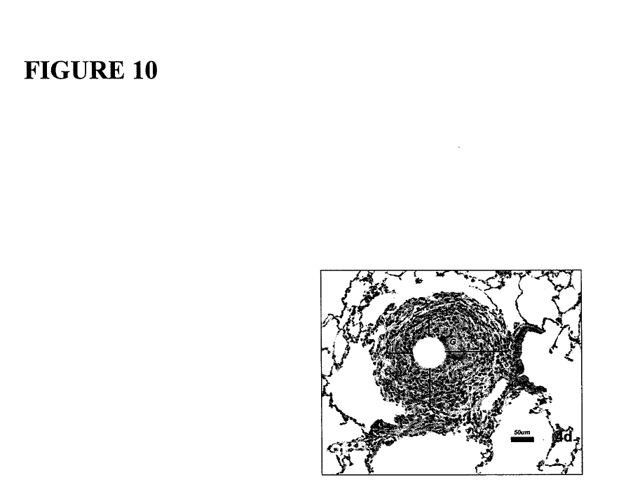

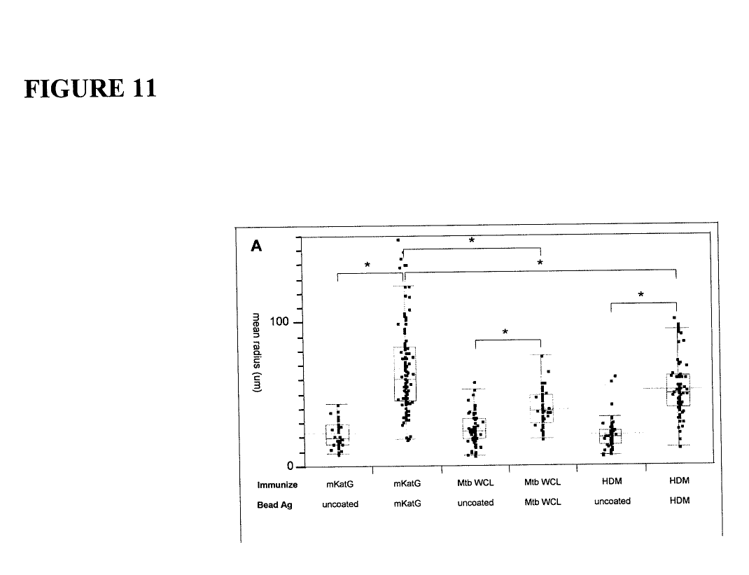

To quantitate the granulomatous inflammation, the radius (extent by distance) of granulomatous inflammation was measured along orthogonal axes and averaged (FIG. 10 ). Comparisons were made with rats exposed to a second Th14d antigen (Mtb whole cell lysate) or Th2 antigen (immunized with Th2-promoting alum plus house dust mite (HDM) and then exposed to HDM coated-beads). mKatG coated beads induced significantly larger granulomas than uncoated beads (FIG. 11 ) (23±9 vs. 67±3 μm, p<0.05). Interestingly, mKatG induced significantly larger granulomas compared to granulomas induced by either a comparison Th1 antigen (Mtb whole cell lysate (Mtb-WCL) or Th2 antigen house dust mite (HDM) (all comparisons, p<0.05).

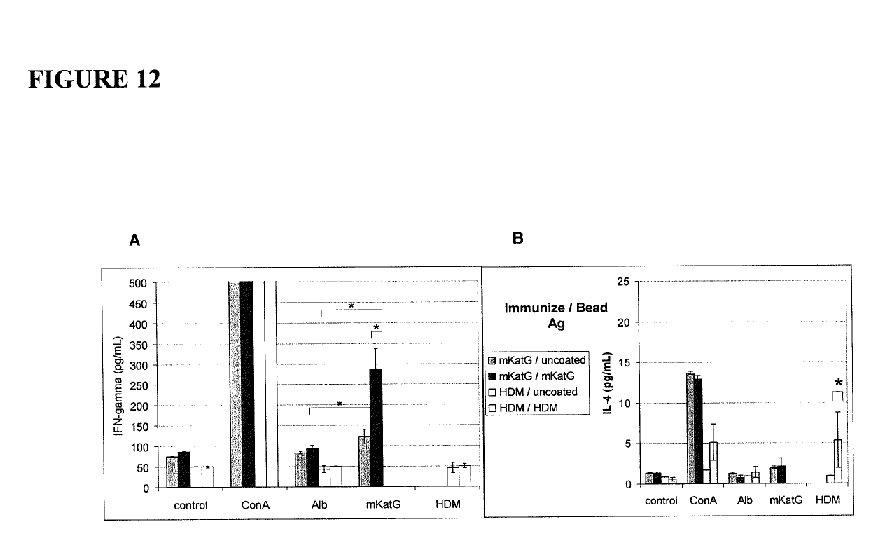

To determine if the granulomatous response to mKatG-beads was associated with Th1 cytokine production, splenocytes were isolated by mechanical dissociation from individual animals 4 days following antigen-coated bead, filtered through 70 μm nylon mesh, and stimulated with mKatG or control antigens for 24 h. Splenocytes from rats immunized to mKatG and then exposed to mKatG-coated beads produced significantly greater levels of IFNγ (FIG. 12 A) than media alone or control antigen (recombinant human albumin) (all comparison, p<0.05). In contrast, splenocytes from rats immunized with HDM produced detectable levels of IL-4 in response to HDM antigen, but not mKatG (FIG. 12 B).

### Example 16

### Murine Model of Sarcoidosis

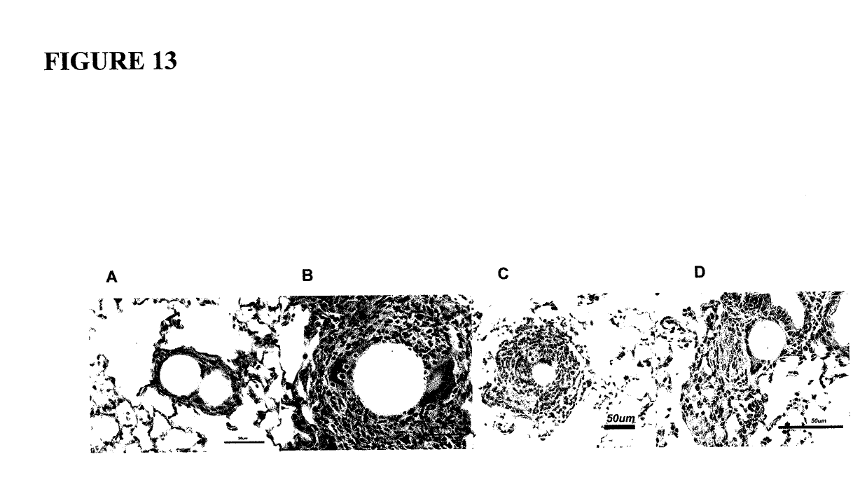

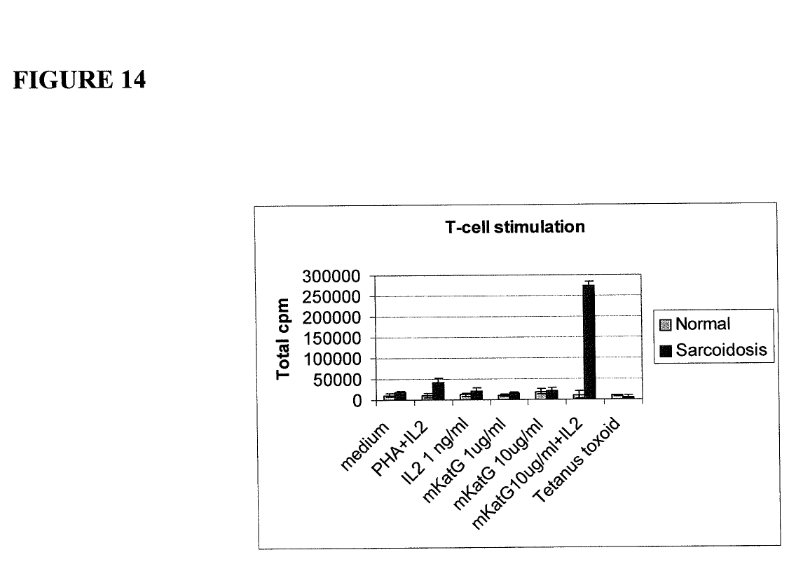

Although mice do not form granulomas as readily as guinea pigs, rabbits or rats when exposed to mycobacterial organisms, given the greater genetic resources available in the mouse, we determined if mKatG-linked beads induces granulomatous inflammation in the mouse. C57BL/6J mice were immunized with mKatG in CFA 14d prior to IV bead injection with Sephadex-linked beads. Similar to the rat, a significant mononuclear cell infiltrate including giant cells was observed around mKatG-coated beads compared to uncoated control beads (FIG. 13 ). This infiltrate was diminished but still present at 10 days (FIG. 13 D) with increased matrix staining suggesting the development of fibrosis, similar to the rat. In contrast, mice immunized with Th2-promoting alum plus HDM and then exposed to HDM coated-beads demonstrated a vigorous eosinophil-dominant mononuclear cell infiltrate (FIG. 14C). Our data suggest mKatG induces larger granulomas than HDM (mKatG vs. HDM, 39±2 vs. 31±3 μm, p<0.073). This is in contrast to published reports that indicate Th2 driven granulomatous inflammation in the mouse is more robust than Th1 driven granulomatous inflammation. Giant cells were more frequently observed in rats, with a frequency of 1-2 per granuloma vs. <1 per granuloma in mice.

### Example 17

### Identification of Candidate Proteins Using MALDI-TOF Mass Spectroscopy

To identify the 62-64 kD proteins that were targets of sarcoid IgG, we combined sarkosyl extracted pellets (Pe) from individual aliquots of a sarcoidosis spleen that demonstrated intense bands in this region (FIG. 2). The combined sample was run on a 4-12% tris-bis Novex gel, stained with Coomassie blue, and bands of approximately 60-65 kD size were excised. The proteins in the bands were digested with trypsin in-situ and the resulting peptides extracted, precipitated under low and high salt conditions, and analyzed by MALDI-TOF mass spectrometry. The peptide mass fingerprint was analyzed by protein database sequence searching using the MOWSE (Molecular Weight Search) database and MS Fit search engine (using Protein Prospector at USC (prospector.ucsfedu) and London websites) and the Mascot search engine (Matrix Science, Ltd at www.matrixscience.com). Unexpectedly, the highest match using Protein Prospector was for M. tuberculosis KatG protein (MW=80625) (MOWSE score 1.07×10e+09 with 18/74 peptide matches for the combined low and high salt peptide solutions (FIG. 3).

To identify other potential candidate antigens and to evaluate whether mycobacterial KatG peptides are found in other sarcoidosis tissues, the 60-65 kD band from two additional sarcoidosis samples were analyzed by MALDI-TOF mass spectrometry. Strikingly, the MALDI-TOF mass spectrum of a 60-65 kD band excised from a sarcoidosis lung (same as in FIG. 2 , Lane 5) demonstrated 4/26 peptide fragments that matched with M. smegmatis KatG (msgKatG) (MOWSE score 1.39×10e03) (Table 1). Preliminary MALDI-TOF analysis of a TX 100-extracted, PK-treated sarcoidosis LN sample with a single 60-65 kD band on an immunoblot with sarcoidosis but not control sera yielded a match to Helicobacter pylori (strain J99) catalase (MW=58490) with 11/28 matching peptide fragments (Mascot score, 58). The fact that three independently processed samples from different sarcoidosis tissues all provided matches for mycobacterial or bacterial catalases provides strong preliminary data that this class of proteins may be involved in the pathogenesis of sarcoidosis.

TABLE I

Summary of peptide matches for M. smegmatis KatG using MALDI-TOF
MS of sarcoidosis tissue

| | | | | | |
|---|---|---|---|---|---|
| m/z | MH+ | Delta | | | |
| submitted | matched | Ppm | start | end | Peptide Sequence |

| | | | | | |
|---|---|---|---|---|---|
| 1709.7000 | 1709.7883 | −51.6653 | 365 | 380 | (K)DNGWANSVPLAHEDGK(T) |
| 1914.0000 | 1913.0530 | 495.0163 | 613 | 631 | (R)ANLLGLSAPEMTTLVGGLR(V) |
| 2061.4000 | 2062.0102 | -295.9038 | 263 | 282 | (R)MAMNDVETAALIVGGHTFGK(T) |
| 2503.3000 | 2502.1425 | 462.6087 | 442 | 463 | (K)DTWLWQDNIPAGNDLSDDEVAK(L) |

### Example 18

### Immunoblot and In Situ Hybridization Analysis Confirms Presence of Mycobacterial KatG Protein and DNA in Sarcoid Tissue

To confirm the presence of mKatG protein in sarcoid tissue, protein immunoblots were probed with two MAbs reactive to M. tuberculosis KatG protein (IT57, IT42 obtained from Colorado State University under NIH, NIAID contract N01 AI-75320). We found that sarcoidosis tissues but not control tissues demonstrated the presence of mycobacterial KatG using these antibodies leading us to the hypothesis that mKatG proteins are pathogenic antigens in sarcoidosis. (FIG. 4). In situ hybridization confirmed that mkatG DNA was present in a subset of sarcoidosis tissues (FIG. 6).

### Example 19

### B Cell Responses to Denatured mKatG Determinants in Sarcoidosis

To confirm that mKatG was a target of circulating IgG in sarcoidosis, the M. tuberculosis katG gene was cloned and the protein expressed and purified for use in protein immunoblots. Anti-mKatG mAbs bound to recombinant M. tuberculosis KatG of the expected wild-type 80 kD size as well as other higher migrating species that were shown to be derived from mKatG by MALDI-TOF mass spectrometry (FIG. 5).

To determine the contribution of individual patient's IgG to the binding to the mKatG proteins, we analyzed binding of individual sarcoidosis sera to soluble mKatG (FIG. 7). IgG antibodies to recombinant mKatG were detected in 12/25 (48%) sarcoidosis patients compared to 0/11 (0%) PPD− (p=0.0059) and 4/10 (40%) PPD+ (p=0.7233) control subjects (FIG. 7). Thus, remnant mycobacterial catalase/peroxidase is a pathogenic tissue antigen that is the target of the adaptive immune responses driving granulomatous inflammation in a subgroup of sarcoidosis patients.

### Example 20

### Sarcoidosis is Associated with T Cell Responses to Recombinant mKatG

IgG responses are generally dependent on T cell help. Since Th1 responses may enhance pathogenic IgG responses, we hypothesized that sarcoidosis would be associated with antigen-specific Th1 immune responses to mKatG proteins. To assess mKatG-specific T cell responses in sarcoidosis and control populations, we first tested T cell proliferation in a standard lymphocyte proliferation assay. Our first sets of sarcoidosis patients and controls have shown a striking proliferative response to mKatG plus IL2 compared to healthy controls (FIG. 14 ). These observations strongly suggest that mKatG induces antigen specific proliferation in sarcoidosis T cells. The dramatic response to mKatG plus 1 ng/ml IL2 (a suboptimal dose) is consistent with a recently stimulated memory T cell population that requires IL2 to avoid cell anergy or apoptosis. Newer techniques have been employed to further examine T cell responses to mKatG using ELISPOT and flow cytometric techniques (below).

Although the molecular details of the T and B cell responses to mKatG in sarcoidosis remain uncertain, it may be relevant that cytokine expression in sarcoidosis appears to be more polarized towards Th1 cytokine expression than in tuberculosis. This finding alone predicts that quantitative and qualitative differences in the humoral and cellular responses to mycobacterial antigens exist between these two disorders, perhaps exemplified by the differences in humoral responses to mKatG determinants detailed above. The persistence of large mycobacterial proteins in tissues may be particularly relevant to long-term immunologic stimulation in sarcoidosis from the slow degradation and release of mycobacterial peptides or from fostering the development of autoimmunity. Persistent bacterial antigens have been convincingly implicated in several autoimmune diseases. For example, persistent Gram-negative bacterial antigens from Yersinia and Salmonella have been linked to reactive arthritis as a result of either antigen persistence or from the development of autoimmunity by molecular mimicry. The possibility that dominant Th1 immune responses may favor a pathogenic humoral response also has precedence in murine models of myasthenia gravis.

### Example 23

### Access Study

Data from the multi-center ACCESS (A Case Control Etiologic Study of Sarcoidosis) study yielded few positive associations with risk of sarcoidosis. However, intriguingly, among those exposures were: workplace insecticide exposure, exposure to mold/mildew at work and exposure to musty odors at work. These exposures clearly could be a marker for exposure to microbes, including mycobacterial organisms. This study was not designed to further define or explore what these exposures meant in terms of microbial agents, but would be consistent with a microbial cause of sarcoidosis. Other findings of the study included confirmation of a familial and likely a genetic basis for the disease, partly explained by association with MHC DR and DQ genes, that smoking is negatively associated with sarcoidosis, and that there is not a single environmental or occupational exposure that is strongly and frequently associated with disease.

Our studies demonstrate the presence of a small number of protease-resistant, neutral detergent-insoluble proteins that by immunoblot analysis are targets of T cell dependent IgG from patients with sarcoidosis but not healthy controls. MALDI-TOF mass spectroscopy and immunoblot analyses have identified KatG proteins from M. tuberculosis and M. smegmatis as two candidate pathogenic antigens. We have data that mKatG induces both T and B cell responses in sarcoidosis, including a possible disease-specific IgG response to denatured mKatG epitopes. T cell responses to mycobacterial hsp65 in sarcoidosis patients provide further evidence of anti-mycobacterial immune responses in sarcoidosis. Other antigens in sarcoidosis tissues, particularly higher molecular weight proteins, remain unidentified.

Based on these observations, we propose that the pathogenesis of sarcoidosis involves T and B cell immune reactivity to denatured or altered microbial proteins that in most cases are mycobacterial in origin and involve the highly expressed KatG proteins. In this scenario, a highly polarized Th1 immune response to mycobacterial organisms in sarcoidosis is accompanied by an ineffective or pathogenic Th2 response, permitting denatured, aggregated or altered mycobacterial proteins to persist in tissues. Mycobacterial protein aggregates either fail to induce a relevant antibody response or bind with IgGs to form insoluble complexes in affected tissues that are not cleared by effective FcR-mediated mechanisms. Proteins in these aggregates induce persistent T cell immunity to peptides derived from their slow degradation by mononuclear phagocytes or induce tissue-specific autoimmunity. In patients with remitting sarcoidosis, a polarized Th1 response is accompanied by a beneficial Th2 response with epitope-specific antibodies that promote clearance of these microbial antigens through FcR-mediated mechanisms. In other subsets of patients, non-mycobacterial organisms such as P. acnes induce disease with a similar pathogenesis. In both cases, altered, denatured microbial protein complexes serve as a nidus for granuloma formation and induce linked Th1 and pathogenic B cell responses to these tissue antigens.

### Example 24

### Sarcoidosis is Triggered by Microbial Antigens: Identification of mKatG as a Candidate Pathogenic Antigen

We used a proteomic approach to identify candidate tissue antigens relevant to immune mediated granulomatous inflammation in sarcoidosis in order to gain insight into the etiopathogenesis of the disease. A limited proteomic approach using protein immunoblot and MALDI-TOF MS to identify candidate tissue antigens was employed, followed by patient studies to validate candidate antigens as targets of the immune response in sarcoidosis. This approach was a general one, without specifying a candidate antigen beforehand (e.g. specific microbial antigen or host autoantigen) other than the nature of its biochemical characteristics. Despite the high risk and labor-intensive requirement of these studies, our results using this approach led to identification of mycobacterial catalase-peroxidase (mKatG) as a pathogenic antigen (Song et al. J Exp Med. (2005); 201:755-67). Circulating mKatG IgG is present in ˜50% of sarcoidosis patients, <10% PPD− controls, and ˜70% PPD+ controls. More recently, we have found evidence for T cell responses in similar proportions in these groups of patients (see below), providing additional support that mKatG is a pathogenic antigen in a subset of sarcoidosis.

### Example 25

### mKatG is a Target of the Adaptive T Cell Response in a Majority of Sarcoidosis Patients

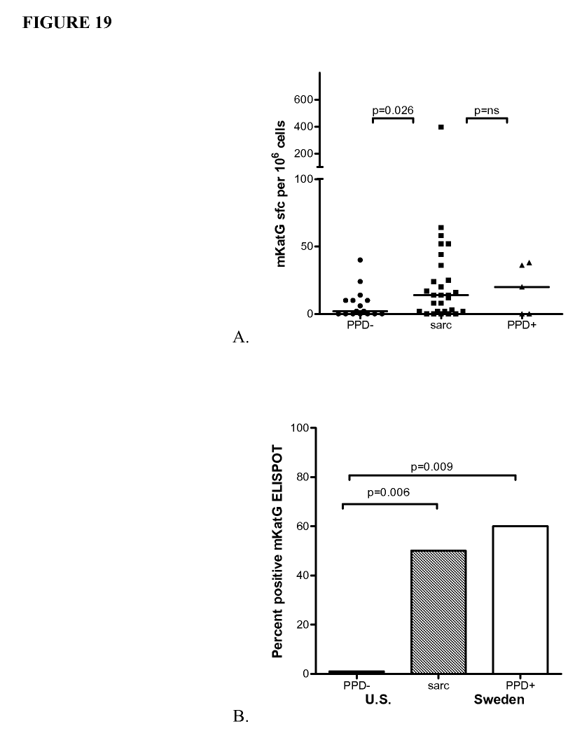

To evaluate antigen specific T cell responses to mKatG in sarcoidosis, we have utilized ELISPOT assays to determine the frequency of mKatG-specific, IFNγ-producing T cells in PBMC and lung (BAL) from patients with sarcoidosis and PPD+ and PPD− healthy controls (FIG. 19). We demonstrate higher proportions of circulating mKatG reactive T cells producing IFNγ in patients with sarcoidosis compared to PPD-healthy control subjects (FIG. 8, 15, 19A). Using stringent criteria to define a positive “mKatG responder” by a subject with mKatG spot forming units (spf)≧2× media control, ≧10 above bkd and with a total>13 mKatG spf (>95% higher than PPD− control subjects), we demonstrate positive IFNγ-producing mKatG specific T cell responses in the blood of ˜50% of sarcoidosis patients, <2% of PPD− controls and ˜60% PPD+ control subjects (FIG. 19B). The frequency of IFNγ-producing PBMC in response to mKatG from sarcoidosis patients was also greater than the frequency of IFNγ-producing PBMC from PPD-negative control subjects.

### Example 26

### Responses of CD4 and CD8 T Cells to mKatG in Sarcoidosis Patients

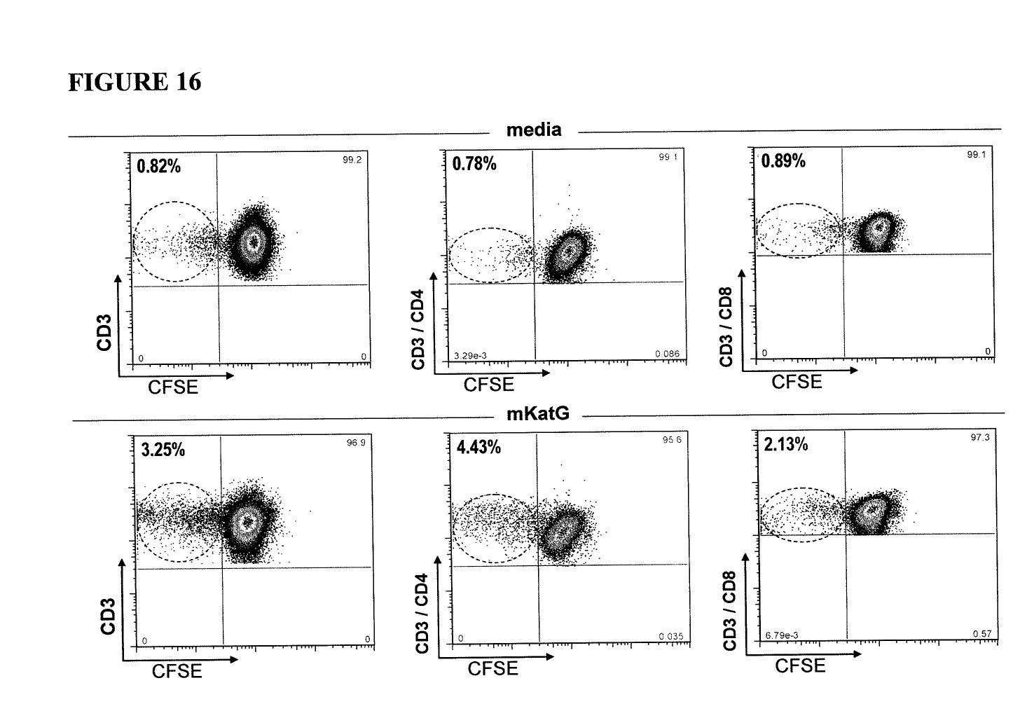

To evaluate the proliferative response and identify the T cell subsets that respond to mKatG in sarcoidosis, we used flow cytometry and CFSE staining. We find that ˜50% of sarcoidosis patients have proliferative responses to mKatG in either lung or blood, with greater proliferative responses in blood than lung, and greater responses in CD4+ compared to CD8+ subsets (FIG. 16, 17).

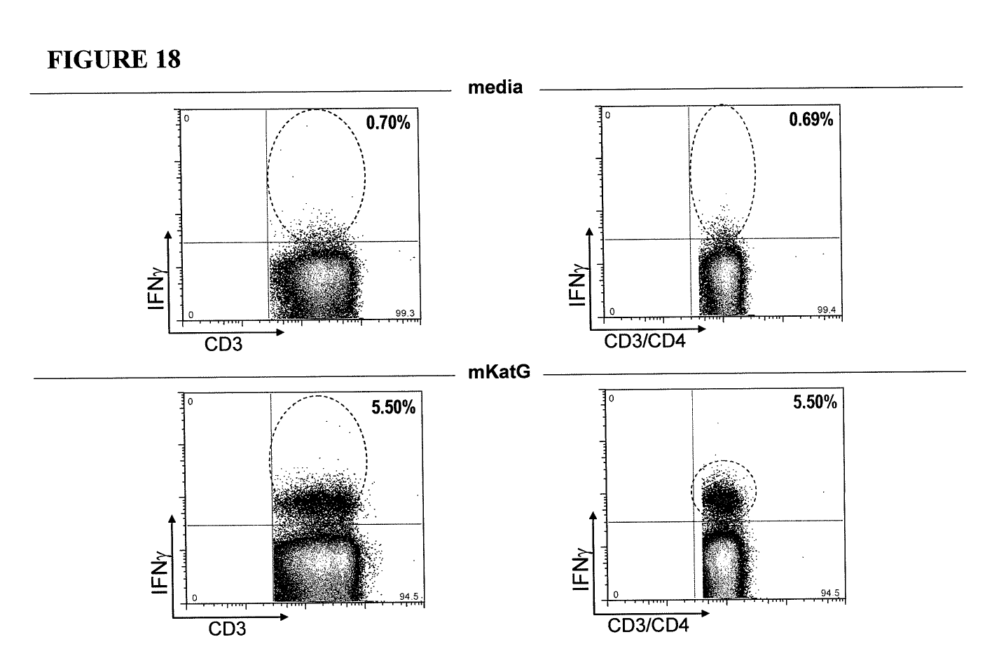

Using intracellular staining techniques, we confirm that mKatG responsive CD3 and CD4 T cells produce IFNγ in lung and blood (FIG. 18 ). Thus, remnant mycobacterial catalase/peroxidase is a pathogenic tissue antigen that is a target of the adaptive T and B cell immune responses driving granulomatous inflammation in a subset of sarcoidosis.

### Example 27

### Overexpression and Purification of Mtb KatG or Synthesis of Protein Derivatives or Variants

Mtb KatG protein is overexpressed from the plasmid construct pYZ56, which contained the katG gene in a 2.9 kDa EcoRV-KpnI fragment in pUC19 vector (Zhang, Y., et al. The catalase-peroxidase gene and isoniazid resistance of Mycobacterium tuberculosis. (1992) Nature 358:591-93). The KatG protein forms a fusion protein with 22 amino acids of the lacZ gene and the overexpression is driven by the lacZ promoter on the pUC19. The pYZ56 construct is transformed into the E. coli catalase mutant UM2 and the transformant is grown in LB medium containing 50 μg/mL ampicillin overnight. Protein extracts are prepared by sonication on ice, and the purification of the KatG protein is performed as described by Johnsson and coworkers (Johnsson, K., et al. Overexpression, purification and characterization of the catalase-peroxidase KatG from Mycobacterium tuberculosis. (1997) J. Biol. Chem. 272:2834-40). Peptides or peptide derivatives and variants are synthesized with common, commercially available technologies and purified by chromatographic techniques. Production and purification of recombinant protein, peptide or peptide derivatives and variants must meet federal standards for use in human subjects.

### Example 29

### In Vivo Diagnostic Testing

Recombinant mKatG will be administered by intradermal injection in concentrations to be determined by clinical studies. The skin test reaction will be assessed at 15 minutes (to assess possible allergic immediate hypersensitivity), at approximately 48-72 hours (to assess for a delayed type hypersensitivity reaction analogous to the PPD skin test for tuberculosis) and at 2-4 weeks (to assess for development of a nodule which will then be biopsied, analogous to the Kveim skin test) (Munro, C. S, and Mitchell, D. N. The Kviem response: still useful, still a puzzle. (1987) Thorax 42:321-31).

### Example 30

### In Vitro Diagnostic Testing

A sample of blood will be tested for immune responses to recombinant mKatG or its protein derivatives by 1) standard protein immunoblot assays using recombinant mKatG (as described in Song, Z., et al., Mycobacterial catalase-peroxidase is a tissue antigen and target of the adaptive immune response in systemic sarcoidosis. J. Exp. Med. (2005) 201(5):755-67. Erratum in: J. Exp. Med. (2005) 202(5):721) or 2) T cell responses by standard ELISPOT or flow cytometric assays to determine antigen specific T cell responses, using recombinant mKatG or derived peptides.

### Example 31

### mKatG Based Vaccine Therapies

Development of mKatG-based vaccine therapies would involve the administration of recombinant mKatG protein or its derived peptides or peptide mimetics by subcutaneous injection or oral administration.

### Example 32

### Higher T Cell Reactivity to mKatG in the Lung than Blood from Patients with Sarcoidosis

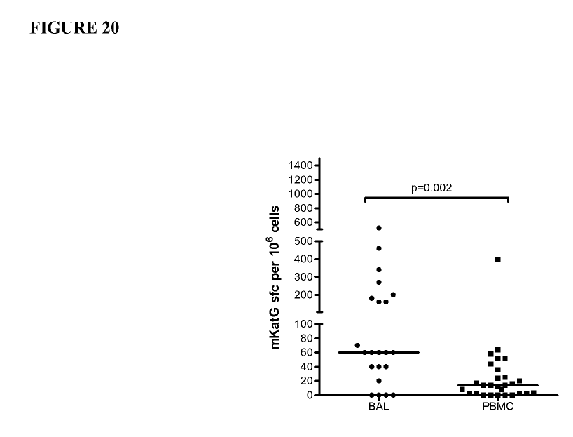

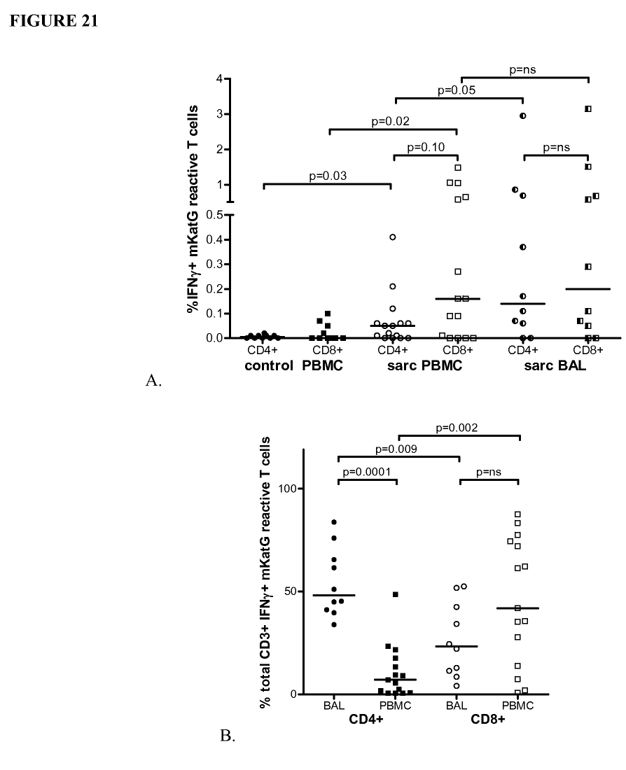

Using ELISPOT assays, we demonstrate that mKatG reactive T cells producing IFNγ accumulate in the lungs of patients with pulmonary sarcoidosis, a site of granulomatous inflammation (FIG. 20). This finding is further confirmed by using intracellular cytokine staining and flow cytometry to determine the proportion of mKatG reactive Th1 cells in lung and blood. We demonstrate the enhanced accumulation of CD3+ CD4+mKatG T cells producing IFNγ in response to mKatG in the lungs of patients with pulmonary sarcoidosis (FIG. 21). These findings are consistent with the concept that mKatG is a pathogenic antigen in sarcoidosis, resulting in accumulation of mKatG reactive Th1 cells (producing IFNγ) at sites of inflammation such as the lung in sarcoidosis.

### Example 33

### T Cell Reactivity to mKatG Decreases with Treatment or Disease Inactivity but not Disease Chronicity

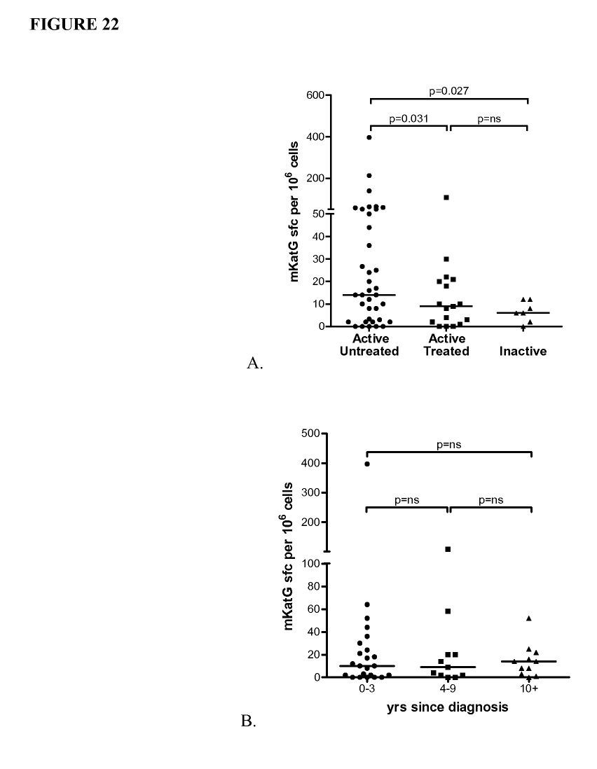

Using ELISPOT assays, we demonstrate that the proportion of mKatG reactive blood T cells producing IFNγ is reduced in patients with sarcoidosis who are under active treatment with corticosteroid or immunosuppressive therapy or in patients who have inactive sarcoidosis with no evidence of disease activity for at least one year (FIG. 22). Furthermore, we demonstrate that the proportion of mKatG reactive blood T cells producing IFNγ does not significantly change despite years of active disease, including those patients with >10 years of known active disease (FIG. 22B). These findings are consistent with the concept that mKatG is a pathogenic antigen in sarcoidosis, with circulating mKatG reactive Th1 cells (producing IFNγ) with persistent disease. Thus, remnant mycobacterial catalase/peroxidase is a pathogenic tissue antigen that is a target of the adaptive T and B cell immune responses driving granulomatous inflammation in a subset of sarcoidosis.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

### REFERENCES

All publications and patents mentioned herein are hereby incorporated by reference in their entirety as if each individual publication or patent was specifically and individually incorporated by reference. In case of conflict, the present application, including any definitions herein, will control.

1. A method for determining whether a subject has or is likely to develop sarcoidosis, comprising
administering to the skin of a subject a microbial catalase or peroxidase protein or antigenic portion thereof, and
determining the presence of a reaction to the microbial catalase or peroxidase or antigenic portion thereof, wherein the presence of a reaction indicates that the subject has or is likely to develop sarcoidosis.

2. The method of claim 1, wherein the reaction is an immune reaction.

3. The method of claim 2, wherein the immune reaction is an immediate hypersensitivity reaction.

4. The method of claim 3, wherein determining the presence of an immediate hypersensitivity reaction is conducted about 5 to 30 minutes after administering a microbial catalase or peroxidase protein or antigenic portion thereof to the skin.

5. The method of claim 2, wherein the immune reaction is a delayed type hypersensitivity reaction.

6. The method of claim 5, wherein determining the presence of a delayed type hypersensitivity reaction is conducted about 48-72 hours after administering a microbial catalase or peroxidase protein or antigenic portion thereof to the skin.

7. The method of claim 1, wherein the reaction is the development of a granulomatous nodule.

8. The method of claim 7, wherein determining the presence of a granulomatous nodule is conducted about 2-4 weeks after contacting the skin with a microbial catalase or peroxidase protein or antigenic portion thereof.

9. The method of claim 3, further comprising determining the presence of a delayed type hypersensitivity reaction and/or the presence of a granulomatous nodule.

10. The method of claim 5, further comprising determining the presence of a granulomatous nodule.

11. The method of claim 1, wherein the microbial catalase or peroxidase protein or antigenic portion thereof is administered to the skin of the subject by intradermal injection.

12. The method of claim 1, wherein the microbial catalase or peroxidase protein is a mycobacterial catalase or peroxidase protein.

13. The method of claim 12, wherein the protein is a Mycobacterium tuberculosis protein.

14. The method of claim 13, wherein the protein is catalase-peroxidase KatG.

15. The method of claim 1, wherein the microbial catalase or peroxidase protein is a Helicobacter pylori catalase.

16. The method of claim 1, wherein the microbial catalase or peroxidase protein is a recombinant protein.

17. A method for determining whether a subject has or is likely to develop sarcoidosis, comprising determining whether the subject has a microbial catalase or peroxidase protein or portion thereof, wherein the presence of a microbial catalase or peroxidase protein or portion thereof and the fact that the subject is not a subject who has or has had tuberculosis or is Purified Protein Derivative (PPD)+, indicates that the subject has or is likely to develop sarcoidosis.

18. A method for determining whether a subject has or is likely to develop sarcoidosis, comprising determining whether the subject has immune cells reactive to a microbial catalase or peroxidase protein, wherein the presence of immune cells reactive to a microbial catalase or peroxidase protein and the fact that the subject is not a subject who has or has had tuberculosis or is Purified Protein Derivative (PPD)+, indicates that the subject has or is likely to develop sarcoidosis.

19. The method of claim 18, comprising obtaining a blood sample from the subject and determining the presence of the immune cells in the blood sample.

20. The method of claim 19, wherein determining the presence of immune cells reactive to a microbial catalase or peroxidase protein comprises contacting immune cells of the subject with a microbial catalase or peroxidase protein or antigenic portion thereof.

21. The method of claim 20, further comprising determining antigen specific T cell responses.

22. The method of claim 21, wherein T cell responses are determined by flow cytometry.

23. A kit for diagnosing sarcoidosis comprising at least one composition comprising a microbial catalase or peroxidase protein or antigenic portion thereof, and optionally instructions for use.

24. A method for treating or preventing sarcoidosis in a subject, comprising administering to a subject in need thereof a tolerizing effective amount of a microbial catalase or peroxidase protein or antigenic portion thereof, such that sarcoidosis is treated or prevented in a subject.

25. The method of claim 24, wherein the tolerizing effective amount of a microbial catalase or peroxidase protein or antigenic portion thereof is administered at about regular intervals for about six months to 3 years.

26. The method of claim 25, wherein the microbial catalase or peroxidase protein or antigenic portion thereof is administered orally to the subject, such as to induce oral tolerance to the protein.

27. A method for treating sarcoidosis in a subject, comprising administering to a subject in need thereof a therapeutically effective amount of a microbial catalase or peroxidase protein or antigenic portion thereof, to induce an immune reaction against the protein, such that sarcoidosis is treated or prevented in a subject.

28. A pharmaceutical composition comprising a tolerizing effective amount of a microbial catalase or peroxidase protein or antigenic portion thereof and a pharmaceutically acceptable vehicle.

29. A pharmaceutical composition comprising a therapeutically effective amount of a microbial catalase or peroxidase protein or antigenic portion thereof and a pharmaceutically acceptable vehicle.


SEQUENCE LISTING (30 sequences)

SEQ ID NO: 1 - protein, 740 aa, Mycobacterium tuberculosis
MPEQHPPITETTTGAASNGCPVVGHMKYPVEGGGNQDWWPNRLNLKVLHQNPAVADPMGAAFDYAAEVATIDVDALTRDIEEVMTTSQPWWPADYGHYGPLFIRMAWHAAGTYRIHDGRGGAGGGMQRFAPLNSWPDNASLDKARRLLWPVKKKYGKKLSWADLIVFAGNCALESMGFKTFGFGFGRVDQWEPDEVYWGKEATWLGDERYSGKRDLENPLAAVQMGLIYVNPEGPNGNPDPMAAAVDIRETFRRMAMNDVETAALIVGGHTFGKTHGAGPADLVGPEPEAAPLEQMGLGWKSSYGTGTGKDAITTGIEVVWTNTPTKWDNSFLEILYGYEWELTKSPAGAWQYTAKDGAGAGTIPDPFGGPGRSPTMLATDLSLRVDPIYERITRRWLEHPEELADEFAKAWYKLIHRDMGPVARYLGPLVPKQTLLWQDPVPAVSHDLVGEAEIASLKSQIRASGLTVSQLVSTAWAAASSFRGSDKRGGANGGRIRLQPQVGWEVNDPDGDLRKVIRTLEEIQESFNSAAPGNIKVSFADLVVLGGCAAIEKAAKAAGHNITVPFTPGRTDASQEQTDVESFAVLEPKADGFRNYLGKGNPLPAEYMLLDKANLLTLSAPEMTVLVGGLRVLGANYKRLPLGVFTEASESLTNDFFVNLLDMGITWEPSPADDGTYQGKDGSRKVKWTGSRVDLVFGSNSELRALVEVYGADDAQPKFVQDFVAAWDKVMNLDRFDVR

SEQ ID NO: 2 - protein, 740 aa, Mycobacterium smegmatis
  MOD_RES (42)..(42): Any amino acid
MPEDRPIEDSPPIGEAQTDAPAGGCPAGFGRIKPPVAGGSNXDWWPNQLNMKILQKNPDVINPLDEDFDYRSAVQNLDVDALRADIVEVMHTSQDWWPADFGHYGPLFIRMAWHAAGTYRVSDGRGGAGAGMQRFAPLNSWPDNASLDKARRLLWPVKKKYGKNLSWADLIVYAGNVALEDMGFRTAGFAFGREDRWEPEEDVYWGPEQEWLDRTKRYTGERDLENPLAAVQMGLIYVNPEGPNGNPDPQASAIDIRETFGRMAMNDVETAALIVGGHTFGKTHGNGDASLVGPEPEAAPLEEVGLGWRNPQGTGVGKDAITSGLEVTWTHTPTKWDNSFLEILYGNEWELTKSPAGANQWKPKDNGWANSVPLAHEDGKTHPSMLTSDLALRVDPIYEQITRRWLDHPEELAEEFAKAWFKLLHRDMGPVTRYLGPEVPKDTWLWQDNIPAGNDLSDDEVAKLKELIADSGLTVSQLVSTAWKAASTFRSSDLRGGANGGRIRLQPQLGWEANEPDELAQVVRKYEEIQKASGINVSFADLVVLGGNVGVEKAAKAAGFDVTVPFTPGRGDATQEETDVDSFAYLEPKADGFRNYLGKGSDLPAEFKLIDRANLLGLSAPEMTTLVGGLRVLDVNHGGTKHGVLTDKPGALTTDFFVNLLDMSTAWKPSPADDGTYIGTDRATGSPKWTGTRVDLVFASNSQLRALAEVYAEDDSKEKFVKDFVAAWTKVMDADRFDVA

SEQ ID NO: 3 - protein, 480 aa, Proprionibacterium acnes
MPIDPSKPSTNTNGSPAPSDEYSLTVGADGPVVLHDAHLIDTLAHFNRENIPERKPHAKGSGAFGHLEVTADVSEYTKASFLQKGRETPMLARFSTVAGELGSPDTWRDVRGFSLKFYTDEGNFDMVGNNTPVFFMRDPMKFPHFIRSQKRLPNSGLRSPNMMYDYWSLSPESAHQVAYLMGPRGIPLSYRTMNGYSSHTYSWVNAEGKITWVKYHFISDQGVHNMTADRAKAIVGDHPDIHREDLFNHIADGDYPSWTVKVQLMPYDEAKDYRFNPFDITKVWPHKDYPLHTIGKFTLDRNPENFFAQIEQAAFSPSNTVEGTGLSPDRMLLGRVFAYNDAARNRLGVNYEQLPVNRPTTPTNQYTFDGQMAFEHSGSAPTYAPNSYGRDFATGYRTGSEATWEADGELVRSAQTRHAEDDDFGQAHALVHDVFSDAERDELVDTLVDLMTNFDMEEPVIGNALSYWRNIDAGIAEATE

SEQ ID NO: 4 - protein, 167 aa, Proprionibacterium acnes
MATTAFMGKPLNTVGDLPQVGSLLPSFTLVKSDLSELRSDELKGKKLVLNIFPSVDTGVCATSVRTFNEKAAGLDDTTVLCVSRDLPFAQARFCGAEGIKNVVVASAFRSHFGKDLGVTLADGPMQHLLARAIVVVDAEGKVTYTQLVDEITTEPDYDAALEAASKA

SEQ ID NO: 5 - protein, 505 aa, Helicobacter pylori
MVNKDVKQTTAFGAPVWDDNNVITAGPRGPVLLQSTWFLEKLAAFDRERIPERVVHAKGSGAYGTFTVTKDITKYTKAKIFSKVGKKTECFFRFSTVAGEKGSADAVRDPRGFAMKYYTEEGNWDLVGNNTPVFFIRDAIKFPDFIHTQKRDPQTNLPNPDMVWDFWSNVPESLYQVTWVMSDRGIPKSFRHMDGFGSHTFSLINAKGERFWVKFHFETMQGVKHLTNEEAAEVRKYDPDSNQRDLFDAIAGGDFPKWKMSIQVMPEEDAKKYRFHPFDVTKIWYLQDYPLMEVGIVELNKNPENYFAEVEQAAFTPANVVPGIGYSPDRMLQGRLFSYGDTHRYRLGVNYPQIPVNRPRCPFHSSSRDGYMQNGYYGSLQNYTPSSLPGYKEDKSARDPKFNLAHIEKEFEVWNWDYRAEDSDYYTQPGDYYRSLPADEKERLYDTIGGSLAHVTHKEIVDKQLEHFKKADPKYAEGVKKALEKHQKMMKDMHAKDMHHMKKKK

SEQ ID NO: 6 - DNA, 2223 bp, Mycobacterium tuberculosis
gtgcccgagcaacacccacccattacagaaaccaccaccggagccgctagcaacggctgtcccgtcgtgggtcatatgaaataccccgtcgagggcggcggaaaccaggactggtggcccaaccggctcaatctgaaggtactgcaccaaaacccggccgtcgctgacccgatgggtgcggcgttcgactatgccgcggaggtcgcgaccatcgacgttgacgccctgacgcgggacatcgaggaagtgatgaccacctcgcagccgtggtggcccgccgactacggccactacgggccgctgtttatccggatggcgtggcacgctgccggcacctaccgcatccacgacggccgcggcggcgccgggggcggcatgcagcggttcgcgccgcttaacagctggcccgacaacgccagcttggacaaggcgcgccggctgctgtggccggtcaagaagaagtacggcaagaagctctcatgggcggacctgattgttttcgccggcaactgcgcgctggaatcgatgggcttcaagacgttcgggttcggcttcggccgggtcgaccagtgggagcccgatgaggtctattggggcaaggaagccacctggctcggcgatgagcgttacagcggtaagcgggatctggagaacccgctggccgcggtgcagatggggctgatctacgtgaacccggaggggccgaacggcaacccggaccccatggccgcggcggtcgacattcgcgagacgtttcggcgcatggccatgaacgacgtcgaaacagcggcgctgatcgtcggcggtcacactttcggtaagacccatggcgccggcccggccgatctggtcggccccgaacccgaggctgctccgctggagcagatgggcttgggctggaagagctcgtatggcaccggaaccggtaaggacgcgatcaccaccggcatcgaggtcgtatggacgaacaccccgacgaaatgggacaacagtttcctcgagatcctgtacggctacgagtgggagctgacgaagagccctgctggcgcttggcaatacaccgccaaggacggcgccggtgccggcaccatcccggacccgttcggcgggccagggcgctccccgacgatgctggccactgacctctcgctgcgggtggatccgatctatgagcggatcacgcgtcgctggctggaacaccccgaggaattggccgacgagttcgccaaggcctggtacaagctgatccaccgagacatgggtcccgttgcgagataccttgggccgctggtccccaagcagaccctgctgtggcaggatccggtccctgcggtcagccacgacctcgtcggcgaagccgagattgccagccttaagagccagatccgggcatcgggattgactgtctcacagctagtttcgaccgcatgggcggcggcgtcgtcgttccgtggtagcgacaagcgcggcggcgccaacggtggtcgcatccgcctgcagccacaagtcgggtgggaggtcaacgaccccgacggggatctgcgcaaggtcattcgcaccctggaagagatccaggagtcattcaactccgcggcgccggggaacatcaaagtgtccttcgccgacctcgtcgtgctcggtggctgtgccgccatagagaaagcagcaaaggcggctggccacaacatcacggtgcccttcaccccgggccgcacggatgcgtcgcaggaacaaaccgacgtggaatcctttgccgtgctggagcccaaggcagatggcttccgaaactacctcggaaagggcaacccgttgccggccgagtacatgctgctcgacaaggcgaacctgcttacgctcagtgcccctgagatgacggtgctggtaggtggcctgcgcgtcctcggcgcaaactacaagcgcttaccgctgggcgtgttcaccgaggcctccgagtcactgaccaacgacttcttcgtgaacctgctcgacatgggtatcacctgggagccctcgccagcagatgacgggacctaccagggcaaggatggcagtcgcaaggtgaagtggaccggcagccgcgtggacctggtcttcgggtccaactcggagttgcgggcgcttgtcgaggtctatggcgccgatgacgcgcagccgaagttcgtgcaggacttcgtcgctgcctgggacaaggtgatgaacctcgacaggttcgacgtgcgctga

SEQ ID NO: 7 - DNA, 2223 bp, Mycobacterium smegmatis
atgcctgaggatcgcccgatcgaagacagcccgcccatcggagaagctcagaccgacgcgcctgcgggtggttgcccggcgggtttcggccgcatcaagcctccggtggccggtggcagcaacsgtgactggtggcccaatcagctcaatatgaagatcctgcagaagaacccggacgtcatcaatccgctggacgaggatttcgactaccggtccgcggttcagaacctcgatgtcgacgcgctgcgcgccgacatcgtcgaggtcatgcacacgtcgcaggactggtggcctgccgacttcggccactacggaccgctgttcatccgcatggcctggcacgccgcgggcacctaccgcgtcagtgacggccgcggcggtgcgggcgcgggcatgcagcgcttcgcgccgctgaacagctggcccgacaacgcgagcctggacaaggcccgccgcctgctgtggccggtcaagaagaagtacggcaagaacctgtcgtgggccgacctgatcgtgtatgccggcaatgtggcgctggaggacatgggcttccgcaccgccggcttcgcgttcggtcgcgaggaccgctgggagcccgaggaggacgtgtactggggtcccgagcaggaatggctcgaccgaacaaagcgctacaccggtgagcgtgacctggagaacccgcttgccgcggtccagatgggcctcatctacgtcaaccccgaaggccccaacggcaatccggatccgcaggcctcggccatcgacatccgcgagacgttcggccggatggcgatgaacgacgtcgagaccgccgcgctgatcgtcggtggccacaccttcggcaagacgcacggcaacggcgacgcctcgctggtgggccccgagccggaagccgccccgctcgaagaggtcggcctcggctggcgcaacccgcagggcaccggcgtcggcaaggacgccatcaccagcggtctcgaggtcacctggacccacacgccgaccaagtgggacaacagcttcctggagatcctgtacggcaacgagtgggagctcaccaagagccccgcgggcgccaaccagtggaagcccaaggacaacggctgggccaactcggtgccgctggcccacgaggacggcaagacccacccgtcgatgctgacctcggatctcgcgctgcgcgtcgacccgatctacgagcagatcacccgtcgctggctggatcaccccgaggaacttgccgaggagttcgccaaggcgtggttcaagctgctgcaccgcgacatgggtccggtgacccgctacctgggccccgaggtgccgaaggacacctggctgtggcaggacaacatcccggcgggcaacgatctgtccgacgacgaggtggccaagctcaaggagctgatcgccgactcgggtctgactgtgtcgcagctggtttcgaccgcgtggaaggccgcgtcgaccttccggtcgagtgacctgcgcggcggcgccaacggcggccgcatccgcctgcagccgcagctgggctgggaggccaacgagcccgacgagctcgcccaggtggtccgcaagtacgaggagatccagaaggcgtccggcatcaacgtgtcgttcgccgacctggtggtcctcggcggcaacgtgggtgtcgagaaggccgccaaggcagccggattcgacgtcaccgtgccgttcacgccgggccgtggcgacgccacccaggaagagaccgacgtcgactcgttcgcctacctggagcccaaggccgacggcttccgcaactacctgggcaagggttcggacctgcccgcggagttcaagctgatcgaccgggcgaacctgctgggcctgtcggctcctgagatgaccacgctcgtcggcggtctgcgggtgctcgacgtcaaccacggcggtaccaagcacggcgtgctgaccgacaagccgggcgcgttgaccacggacttcttcgtcaacctgctcgacatgtccaccgcatggaagccctcgcctgccgatgacggcacctacatcggcaccgaccgtgccaccggttcgcccaagtggaccggcacgcgtgtcgatctggtgttcgcgtcgaactcgcagctgcgggccctggccgaggtgtacgcggaggatgactccaaggagaagttcgtcaaggacttcgtcgccgcgtggaccaaggtgatggacgccgaccgcttcgacgtcgcctga

SEQ ID NO: 8 - DNA, 1452 bp, Proprionibacterium acnes
atgcctattgatcccagcaagccgtcaacgaacaccaacgggtcgccagcgcctagcgacgaatactccctgaccgtcggcgctgacggcccggtagtgctccacgacgcacatctcatcgacaccctggcccatttcaaccgggaaaacatccccgaacgcaagcctcacgccaagggatcgggggccttcggacatctcgaggttaccgctgacgtctcggagtacaccaaggcatccttcctgcagaaggggcgcgaaacccccatgctggcccgattctccacagtcgccggtgagctcggcagccctgacacctggcgtgatgtgcgtggattctcgttgaagttctacaccgacgagggcaacttcgacatggtcggcaacaacaccccggtgttcttcatgcgtgacccgatgaagttcccgcatttcatccgctcccagaaacggttaccgaattccggactgcgcagtccgaatatgatgtatgactattggagcctgtcccccgaatcggcccatcaagtggcttacctcatgggaccgcgcggcattccgctcagctatcgcactatgaacggctactcctcgcacacctactcatgggttaacgccgaagggaagatcacctgggtgaaataccacttcatcagtgaccagggtgttcacaacatgactgctgaccgggccaaagcgatcgtcggtgatcacccagacatccaccgcgaggacctcttcaaccacattgccgacggcgactatccgtcctggaccgtcaaggtgcagctcatgccctatgacgaagccaaggattaccggttcaacccgttcgacatcaccaaggtgtggccgcataaggattacccgctgcacaccatcggcaagttcactctggaccgcaacccggagaacttcttcgcccagatcgagcaagcggccttctccccgtcgaacaccgttgagggcactgggttgtccccagaccggatgctgctgggtcgtgtcttcgcctacaacgatgccgcccgcaaccgtctgggggtcaactatgaacaactccctgtgaaccggccaacgacgccgacaaaccagtacaccttcgacggccagatggccttcgaacactccggttcggcacccacctacgccccgaactcctacggtcgcgattttgccaccggataccgaaccggttccgaagctacctgggaggctgacggcgaactcgttcgttccgcccagacccggcacgcggaagatgacgacttcggtcaggcccacgccctggtgcatgacgtcttttccgacgcagagcgtgacgagctcgtcgatactttggtggacttgatgaccaatttcgacatggaagagccggtcatcggcaacgcgctgtcctactggcgcaacatcgacgccggtattgccgaggcaaccgagggcagaatctga

SEQ ID NO: 9 - DNA, 504 bp, Proprionibacterium acnes
atggctactaccgctttcatgggaaaaccccttaacaccgtcggggaccttcctcaggtcggttccttgctgccgtcgttcaccctcgtcaagagtgacttgtcagagctgagaagcgatgagttgaagggcaagaagctcgtcctcaacattttcccaagtgtggacaccggggtgtgtgcaacgagtgtgcgcaccttcaacgagaaagccgctggactcgacgacaccaccgtcttatgtgtttctcgtgaccttccattcgcccaggctcgcttctgcggtgcggagggcatcaagaacgttgttgttgcctcagccttccgctcccattttggtaaggaccttggcgttaccctcgccgatggcccgatgcagcatctgcttgcgcgcgccatcgtcgtcgtggacgctgagggaaaggttacctacacccagctcgttgatgagatcactaccgagccggattacgacgctgcccttgaggctgcgtccaaggcctga

SEQ ID NO: 10 - skipped

SEQ ID NO: 11 - protein, 18 aa, Artificial Sequence
  Description of Artificial Sequence: Synthetic peptide
  MOD_RES (1)..(1): May or may not be present
  MOD_RES (18)..(18): May or may not be present
KDNGWANSVPLAHEDGKT

SEQ ID NO: 12 - protein, 21 aa, Artificial Sequence
  Description of Artificial Sequence: Synthetic peptide
  MOD_RES (1)..(1): May or may not be present
  MOD_RES (21)..(21): May or may not be present
RANLLGLSAPEMTTLVGGLRV

SEQ ID NO: 13 - protein, 22 aa, Artificial Sequence
  Description of Artificial Sequence: Synthetic peptide
  MOD_RES (1)..(1): May or may not be present
  MOD_RES (22)..(22): May or may not be present
RMAMNDVETAALIVGGHTFGKT

SEQ ID NO: 14 - protein, 24 aa, Artificial Sequence
  Description of Artificial Sequence: Synthetic peptide
  MOD_RES (1)..(1): May or may not be present
  MOD_RES (24)..(24): May or may not be present
KDTWLWQDNIPAGNDLSDDEVAKL

SEQ ID NO: 15 - protein, 7 aa, Artificial Sequence
  Description of Artificial Sequence: Synthetic peptide
  MOD_RES (1)..(1): May or may not be present
  MOD_RES (7)..(7): May or may not be present
RGSDKRG

SEQ ID NO: 16 - protein, 9 aa, Artificial Sequence
  Description of Artificial Sequence: Synthetic peptide
  MOD_RES (1)..(1): May or may not be present
  MOD_RES (9)..(9): May or may not be present
KVKWTGSRV

SEQ ID NO: 17 - protein, 12 aa, Artificial Sequence
  Description of Artificial Sequence: Synthetic peptide
  MOD_RES (1)..(1): May or may not be present
  MOD_RES (12)..(12): May or may not be present
RMAWHAAGTYRI

SEQ ID NO: 18 - protein, 16 aa, Artificial Sequence
  Description of Artificial Sequence: Synthetic peptide
  MOD_RES (1)..(1): May or may not be present
  MOD_RES (16)..(16): May or may not be present
KAAGHNITVPFTPGRT

SEQ ID NO: 19 - protein, 16 aa, Artificial Sequence
  Description of Artificial Sequence: Synthetic peptide
  MOD_RES (1)..(1): May or may not be present
  MOD_RES (16)..(16): May or may not be present
RALVEVYGADDAQPKF

SEQ ID NO: 20 - protein, 19 aa, Artificial Sequence
  Description of Artificial Sequence: Synthetic peptide
  MOD_RES (1)..(1): May or may not be present
  MOD_RES (19)..(19): May or may not be present
KAAKAAGHNITVPFTPGRT

SEQ ID NO: 21 - protein, 16 aa, Artificial Sequence
  Description of Artificial Sequence: Synthetic peptide
  MOD_RES (1)..(1): May or may not be present
  MOD_RES (16)..(16): May or may not be present
RWLEHPEELADEFAKA

SEQ ID NO: 22 - protein, 17 aa, Artificial Sequence
  Description of Artificial Sequence: Synthetic peptide
  MOD_RES (1)..(1): May or may not be present
  MOD_RES (17)..(17): May or may not be present
RMAWHAAGTYRIHDGRG

SEQ ID NO: 23 - protein, 17 aa, Artificial Sequence
  Description of Artificial Sequence: Synthetic peptide
  MOD_RES (1)..(1): May or may not be present
  MOD_RES (17)..(17): May or may not be present
KYPVEGGGNQDWWPNRL

SEQ ID NO: 24 - protein, 17 aa, Artificial Sequence
  Description of Artificial Sequence: Synthetic peptide
  MOD_RES (1)..(1): May or may not be present
  MOD_RES (17)..(17): May or may not be present
RRWLEHPEELADEFAKA

SEQ ID NO: 25 - protein, 19 aa, Artificial Sequence
  Description of Artificial Sequence: Synthetic peptide
  MOD_RES (1)..(1): May or may not be present
  MOD_RES (19)..(19): May or may not be present
RFAPLNSWPDNASLDKARA

SEQ ID NO: 26 - protein, 20 aa, Artificial Sequence
  Description of Artificial Sequence: Synthetic peptide
  MOD_RES (1)..(1): May or may not be present
  MOD_RES (20)..(20): May or may not be present
RTLEEIQESFNSAAPGNIKV

SEQ ID NO: 27 - protein, 20 aa, Artificial Sequence
  Description of Artificial Sequence: Synthetic peptide
  MOD_RES (1)..(1): May or may not be present
  MOD_RES (20)..(20): May or may not be present
RNYLGKGNPLPAEYILLDKA

SEQ ID NO: 28 - protein, 23 aa, Artificial Sequence
  Description of Artificial Sequence: Synthetic peptide
  MOD_RES (1)..(1): May or may not be present
  MOD_RES (23)..(23): May or may not be present
KLSWADLIVFAGNCALESMGFKT

SEQ ID NO: 29 - protein, 26 aa, Artificial Sequence
  Description of Artificial Sequence: Synthetic peptide
  MOD_RES (1)..(1): May or may not be present
  MOD_RES (26)..(26): May or may not be present
RGGAGGGMQRFAPLNSWPDNASLDKA

SEQ ID NO: 30 - protein, 24 aa, Artificial Sequence
  Description of Artificial Sequence: Synthetic peptide
  MOD_RES (1)..(1): May or may not be present
  MOD_RES (24)..(24): May or may not be present
KKLSWADLIVFAGNCALESMGFKT